(19) United States
(12) Patent Application Publication
(10) Pub. No.: US 2022/0233174 A1
(43) Pub. Date: Jul. 28, 2022

(54) ULTRASOUND DEVICE CIRCUITRY INCLUDING PHASE-LOCKED LOOP CIRCUITRY AND METHODS OF OPERATING THE SAME

(71) Applicant: BFLY Operations, Inc., Guilford, CT (US)

(72) Inventor: Sewook Hwang, Branford, CT (US)

(73) Assignee: BFLY Operations, Inc., Guilford, CT (US)

(21) Appl. No.: 17/586,656

(22) Filed: Jan. 27, 2022

Related U.S. Application Data
(60) Provisional application No. 63/142,993, filed on Jan. 28, 2021.

(51) Int. Cl. A61B 8/00; H03L 7/099; H03L 7/08

(52) U.S. Cl. CPC A61B 8/54; A61B 8/4477; H03L 7/0802; H03L 7/0991

(57) ABSTRACT

Aspects of the technology described herein relate to an ultrasound device that may has a phase-locked loop (PLL) that includes a digitally-controlled oscillator (DCO). The DCO includes a plurality of current source unit cells with respective drain switches a plurality of current source unit cells with respective source switches. The plurality of current source unit cells with respective drain switches and the plurality of current source unit cells may have different circuit topologies. Switching on one of the plurality of current source unit cells with respective drain switches may cause a voltage transition at an internal node proceeding in one voltage direction and switching on one of the plurality of current source unit cells with respective source switches may cause a voltage transition at an internal node proceeding in the opposite voltage direction.

### CROSS-REFERENCE TO RELATED APPLICATIONS

The present application claims the benefit under 35 U.S.C. § 119(e) of U.S. Patent Application Ser. No. 63/142,993, filed Jan. 28, 2021 under Attorney Docket No. B1348.70191US00, and entitled “ULTRASOUND DEVICE CIRCUITRY INCLUDING PHASE-LOCKED LOOP CIRCUITRY AND METHODS OF OPERATING THE SAME,” which is hereby incorporated by reference herein in its entirety.

### FIELD

Generally, the aspects of the technology described herein relate to circuitry in ultrasound devices and methods of operating such circuitry. Certain aspects relate to phase-locked loop circuitry and methods of operating such circuitry.

### BACKGROUND

Ultrasound probes may be used to perform diagnostic imaging and/or treatment, using sound waves with frequencies that are higher than those audible to humans. Ultrasound imaging may be used to see internal soft tissue body structures. When pulses of ultrasound are transmitted into tissue, sound waves of different amplitudes may be reflected back towards the probe at different tissue interfaces. These reflected sound waves may then be recorded and displayed as an image to the operator. The strength (amplitude) of the sound signal and the time it takes for the wave to travel through the body may provide information used to produce the ultrasound image. Many different types of images can be formed using ultrasound devices. For example, images can be generated that show two-dimensional cross-sections of tissue, blood flow, motion of tissue over time, the location of blood, the presence of specific molecules, the stiffness of tissue, or the anatomy of a three-dimensional region.

### SUMMARY

According to one aspect of the present application, an ultrasound device comprises a phase-locked loop (PLL) comprising a digitally-controlled oscillator (DCO). The DCO comprises a plurality of current source unit cells with drain switch and a plurality of current source unit cells with source switch.

In some embodiments, the plurality of current source unit cells with drain switch have a first circuit topology, the plurality of current source unit cells with source switch have a second circuit topology, and the first and second circuit topologies are different.

In some embodiments, the PLL is configured to use a fast switching technique that allows the PLL to power down when the ultrasound device is not generating data and to power up within 1 microsecond from when the ultrasound device begins to generate data again

In some embodiments, switching on one of the plurality of current source unit cells with drain switch causes a voltage transition at an internal node of the current source unit cell with drain switch proceeding in a first voltage direction, switching on of the plurality of current source unit cells with source switch causes a voltage transition at an internal node of the current source unit cell with source switch proceeding in a second voltage direction, and the first and second voltage directions are opposite.

In some embodiments, each of the plurality of current source unit cells with drain switch comprises a switch and a current source. The current source comprises a first terminal and a second terminal; and the switch is coupled to the first terminal of the current source; and each of the plurality of current source unit cells with source switch comprises the switch; and the current source. The switch is coupled to the second terminal of the current source.

In some embodiments, the current source comprises a single transistor, the first terminal comprises a drain of the transistor, and the second terminal comprises a source of the transistor.

In some embodiments, the current source comprises a cascode current source that comprises multiple transistors, the first terminal comprises a drain of one of the multiple transistors, and the second terminal comprises a source of the one of the multiple transistors.

In some embodiments, the switch comprises a single transistor.

In some embodiments, the switch comprises a transmission gate.

In some embodiments, the current source comprises at least one transistor having a gate terminal, and the gate terminal is coupled to an output of a resistor-capacitor (RC) filter.

In some embodiments, the RC filter is coupled to an output terminal of bias generation circuitry.

In some embodiments, the switch comprises at least one transistor having a gate terminal; the PLL comprises a decoder having a plurality of output terminals; and the gate terminal of the transistor is coupled to one of the plurality of output terminals of the decoder.

In some embodiments, the DCO comprises a ring oscillator; and each of the plurality of current source unit cells with drain switch and each of the plurality of current source unit cells with source switch is couplable to the ring oscillator.

In some embodiments, the ring oscillator is configured to generate a clock signal having a frequency that depends on an amount of current that the ring oscillator receives from the plurality of current source unit cells with drain switch and the plurality of current source unit cells with source switch.

In some embodiments, the ultrasound device is configured to control the frequency of the clock signal, at least in part, by switching on a certain number of the plurality of current source unit cells with drain switch and a certain number of the plurality of current source unit cells with source switch.

In some embodiments, the ultrasound device further comprises control circuitry comprising coarse control circuitry configured to control how many blocks of one or more of the plurality of current source unit cells with drain switch and/or one or more of the plurality of current source unit cells with source switch are turned on.

In some embodiments, each of the plurality of current source unit cells with drain switch and each of the plurality of current source unit cells with source switch is coupled to an output terminal of a resistor-capacitor (RC) filter. A ratio between a decrease in voltage at the output terminal of the RC filter caused by one of the plurality of current source unit cell with drain switch switching on to an increase in voltage at the output terminal of the RC filter caused by one of the current source unit cell with source switch switching on is m:n. Each or approximately each of the blocks comprises 1+floor (n/m) current source unit cells with a composition of floor (n/m) current source unit cells with drain switch and one current source unit cell with source switch.

According to another aspect of the present application, a method, comprise using an ultrasound device comprising a phase-locked loop (PLL). The PPL comprises a digitally-controlled oscillator (DCO) comprising a plurality of current source unit cells with drain switch; and a plurality of current source unit cells with source switch. Using the ultrasound device comprises switching on a certain number of the plurality of current source unit cells with drain switch and a certain number of the plurality of current source unit cells with source switch.

In some embodiments, the plurality of current source unit cells with drain switch have a first circuit topology, the plurality of current source unit cells with source switch have a second circuit topology, and the first and second circuit topologies are different.

In some embodiments, the PLL is configured to use a fast switching technique that allows the PLL to power down when the ultrasound device is not generating data and to power up within 1 microsecond when the ultrasound device begins to generate data again.

In some embodiments, switching on one of the plurality of current source unit cells with drain switch causes a voltage transition at an internal node of the current source unit cell with drain switch proceeding in a first voltage direction, switching on of the plurality of current source unit cells with source switch causes a voltage transition at an internal node of the current source unit cell with source switch proceeding in a second voltage direction, and the first and second voltage directions are different.

In some embodiments, each of the plurality of current source unit cells with drain switch comprises a switch and a current source. The current source comprises a first terminal and a second terminal; and the switch is coupled to the first terminal of the current source; and

each of the plurality of current source unit cells with source switch comprises the switch and the current source. The switch is coupled to the second terminal of the current source.

In some embodiments, the current source comprises a single transistor, the first terminal comprises a drain of the transistor, and the second terminal comprises a source of the transistor.

In some embodiments, the current source comprises a cascode current source that comprises multiple transistors, the first terminal comprises a drain of one of the multiple transistors, and the second terminal comprises a source of the one of the multiple transistors.

In some embodiments, the switch comprises a single transistor.

In some embodiments, the switch comprises a transmission gate.

In some embodiments, the current source comprises at least one transistor having a gate terminal, and the gate terminal is coupled to an output of a resistor-capacitor (RC) filter.

In some embodiments, the RC filter is coupled to an output terminal of bias generation circuitry.

In some embodiments, the switch comprises at least one transistor having a gate terminal; the PLL comprises a decoder having a plurality of output terminals; and the gate terminal of the transistor is coupled to one of the plurality of output terminals of the decoder.

In some embodiments, the DCO comprises a ring oscillator; and each of the plurality of current source unit cells with drain switch and each of the plurality of current source unit cells with source switch is couplable to the ring oscillator.

In some embodiments, using the ultrasound device comprises using the ring oscillator is configured to generate a clock signal having a frequency that depends on an amount of current that the ring oscillator receives from the plurality of current source unit cells with drain switch and the plurality of current source unit cells with source switch

In some embodiments, using the ultrasound device comprises controlling the frequency of the clock signal, at least in part, by switching on the certain number of the plurality of current source unit cells with drain switch and the certain number of the plurality of current source unit cells with source switch.

In some embodiments, the ultrasound device further comprises control circuitry comprising coarse control circuitry. Using the ultrasound device comprises using the coarse control circuitry to switch on or off of blocks comprising one or more of the plurality of current source unit cells with drain switch and or more of the plurality of current source unit cells with source switch at a time.

In some embodiments, each of the plurality of current source unit cells with drain switch and each of the plurality of current source unit cells with source switch is coupled to an output terminal of a resistor-capacitor (RC) filter; the RC filter is coupled to an output terminal of bias generation circuitry; a ratio between a decrease in voltage at the output terminal of the RC filter caused by one of the plurality of current source unit cell with drain switch switching on to an increase in voltage at the output terminal of the RC filter caused by one of the current source unit cell with source switch 114b switching on is m:n; each or approximately each of the blocks comprises 1+floor (n/m) current source unit cells with a composition of floor (n/m) current source unit cells with drain switch and one current source unit cell with source switch.

According to another aspect of the present application, an ultrasound device comprises a plurality of ultrasound transducers, serializer-deserializer (SerDes) circuitry coupled to the plurality of ultrasound transducers, and a phase-locked loop (PLL) comprising a plurality of current source unit cells of a first type and a plurality of current source unit cells of a second type different from the first type.

In some embodiments, at least one of the plurality of current source unit cells of the first type is configured to produce a first voltage increase when turned on, and wherein at least one of the plurality of current source unit cells of the second type is configured to produce a first voltage decrease when turned on.

In some embodiments, the at least one of the plurality of current source unit cells of the first type is configured to produce a second voltage decrease when turned off, and wherein the at least one of the plurality of current source unit cells of the second type is configured to produce a second voltage increase when turned off.

In some embodiments, at least one of the plurality of current source unit cells of the first type comprises a drain switch and at least one of the plurality of current source unit cells of the second type comprises a source switch.

In some embodiments, the source switch comprises a transmission gate.

In some embodiments, the source switch comprises a cascode current source.

According to another aspect of the present application, a method for operating an ultrasound device comprises producing a plurality of electric signals using a plurality of ultrasound transducers and combining the plurality of electric signals using serializer-deserializer (SerDes) circuitry. The combining comprises timing the SerDes circuitry using a phase-locked loop (PLL) at least in part by turning on a plurality of current source unit cells of a first type and at least in part by turning on a plurality of current source unit cells of a second type different from the first type.

In some embodiments, turning on the plurality of current source unit cells of the first type and turning on the plurality of current source unit cells of the second type comprises determining how many current source unit cells of the first type and how many current source unit cells of the second type should be turned on to limit a voltage disturbance.

In some embodiments, determining how many current source unit cells of the first type and how many current source unit cells of the second type should be turned on to limit the voltage disturbance comprises identifying a plurality of entries of a look-up table, that, collectively, yield a voltage disturbance below a predefined threshold.

Some aspects of the present application include at least one non-transitory computer-readable storage medium storing processor-executable instructions that, when executed by at least one processor, cause the at least one processor to perform the above aspects and embodiments. Some aspects include an apparatus having a processing device configured to perform the above aspects and embodiments.

### BRIEF DESCRIPTION OF THE DRAWINGS

Various aspects and embodiments will be described with reference to the following exemplary and non-limiting figures. It should be appreciated that the figures are not necessarily drawn to scale. Items appearing in multiple figures are indicated by the same or a similar reference number in all the figures in which they appear.

FIG. 1 illustrates a schematic block diagram of circuitry in an ultrasound device, in accordance with certain embodiments described herein.

FIGS. 2-6 are circuit diagrams illustrating various examples of current source unit cells, in accordance with certain embodiments described herein.

FIG. 7 illustrates another schematic block diagram of circuitry in an ultrasound device, in accordance with certain embodiments described herein.

FIGS. 8-13 are circuit diagrams illustrating additional examples of current source unit cells, in accordance with certain embodiments described herein.

FIG. 14 is a plot illustrating representative voltage disturbances corresponding to different data sets, in accordance with certain embodiments described herein.

FIG. 15 illustrates a schematic block diagram of an ultrasound device, in accordance with certain embodiments described herein.

### DETAILED DESCRIPTION

In some embodiments, an ultrasound device may include phase-locked loop (PLL) circuitry configured to generate a high-speed clock signal. For example, a PLL may provide the high-speed clock signal to serializer-deserializer (SerDes) circuitry, and the SerDes circuitry may be configured to use the high-speed clock signal to serialize and deserialize data signals. The data signals may be generated and processed by ultrasound sensors and receive circuitry in the ultrasound device.

In some embodiments, the PLL may be configured to generate the clock signal such that the clock signal has a frequency that can be digitally controlled. In particular, in some embodiments, the frequency may depend on the amount of current that a circuit in the PLL, a ring oscillator, receives from current source unit cells. The PLL may be configured to turn on a specific number of these current source unit cells to control the amount of current received by the ring oscillator and thereby control, at least in part, the frequency of the output clock signal.

In some embodiments, the PLL may use fast switching techniques that allow the PLL to power down and conserve power when the ultrasound device is not generating data, and power up to full operation within an acceptably fast period of time (e.g., within 1 microsecond) when the ultrasound device begins to generate data again. The inventors have recognized that when a current source unit cell switches on and off using such a fast switching technique, the current source unit cell may experience a large voltage transition in an internal node that may couple to one or more other nodes in the circuitry of the ultrasound device. The combined effect of all voltage transitions at all the current source unit cells may couple to a node supplying a bias voltage to the PLL, and this large disturbance may cause an increase in the settling time of the voltage at the bias node. If the voltage at this node does not settle sufficiently quickly, then the frequency of the output clock signal from the PLL may not settle in an acceptably short time period.

The inventors have realized that using two types of current source unit cells with two different circuit topologies may help to reduce the disturbance caused by fast switching of the current source unit cells. The topologies may be such that switching on of one type of current source unit cell may cause a voltage transition that proceeds in one voltage direction (e.g., a voltage increase) while switching on of the other type of current source unit cell may cause a voltage transition that proceeds in the opposite voltage direction (e.g., a voltage decrease). Additionally, or alternatively, the topologies may be such that switching off of one type of current source unit cell may cause a voltage transition that proceeds in one voltage direction (e.g., a voltage increase) while switching off of the other type of current source unit cell may cause a voltage transition that proceeds in the opposite voltage direction (e.g., a voltage decrease). Thus, the voltage transitions of multiple current source unit cells of the two types may cancel each other out, or at least attenuate any attenuate the total voltage disturbance. The total disturbance that may couple to the bias voltage node may therefore be less than if all the current source unit cells were of a single type and all the voltage transitions proceeded in the same direction.

In some embodiments, the PLL may include circuitry configured to turn on current source unit cells in blocks of multiple current source unit cells. The inventors have recognized that selecting a specific number of current source unit cells of each type in each block, and selecting a specific order in which current source unit cells turn on or off, may help to reduce the disturbance at the bias voltage node across all options for how many current source unit cells are turned on.

In the below description, a particular reference name (e.g., Vₒᵤₜ, V_{bias}, Vᵢₙₜ, etc.) may refer either to a node of a circuit and/or the particular voltage at that node. As referred to herein, two elements (e.g., terminals, circuits, etc.) being “coupled” together may mean that the two elements are directly coupled together or that another element (e.g., a voltage buffer) is coupled between the two elements.

Various aspects of the present disclosure may be used alone, in combination, or in a variety of arrangements not specifically described in the embodiments described in the foregoing and is therefore not limited in its application to the details and arrangement of components set forth in the foregoing description or illustrated in the drawings. For example, aspects described in one embodiment may be combined in any manner with aspects described in other embodiments.

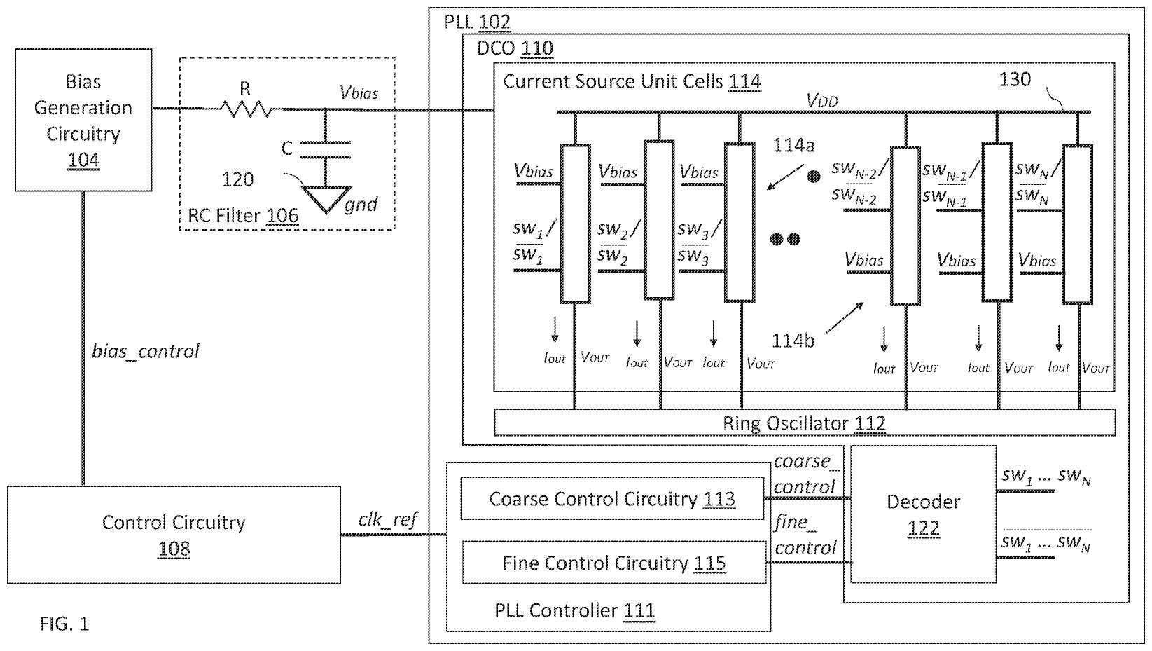

FIG. 1 illustrates a schematic block diagram of circuitry in an ultrasound device, in accordance with certain embodiments described herein. The circuitry in the ultrasound device includes a phase-locked loop (PLL) 102, bias generation circuitry 104, a resistor-capacitor (RC) filter 106, and control circuitry 108. The PLL 102 includes a digitally-controlled oscillator (DCO) 110 and PLL controller 111. The DCO 110 includes a ring oscillator 112, current source unit cells 114, and a decoder 122. The current source unit cells 114 include current source unit cells with drain switch 114a and current source unit cells with source switch 114b. The PLL controller 111 includes coarse control circuitry 113 and fine control circuitry 115. The RC filter 106 includes a resistor R, a capacitor C, and ground 120.

In some embodiments, the PLL 102 may be configured to generate a high-speed clock signal. For example, the PLL 102 may provide the high-speed clock signal to serializer-deserializer (SerDes) circuitry (not illustrated), and the SerDes circuitry may be configured to use the high-speed clock signal to serialize and deserialize data signals. The data signals may be generated and processed by ultrasound sensors and receive circuitry in the ultrasound device. Generally, the PLL 102 includes the DCO 110, which may be configured to generate a clock signal having a frequency that can be digitally controlled, the PLL controller 111, as well as other circuitry (not illustrated). The DCO 110, the PLL controller 111, and the other circuitry in the PLL 102 may be arranged in a feedback loop (not illustrated) configured to force the output high-speed clock signal from the PLL 102 to have the same phase as an input signal to the PLL 102 (clk_ref as illustrated in FIG. 1) and a frequency that depends on the input signal (e.g., a multiple of the frequency of the input signal clk_ref).

As described above, in some embodiments, the DCO 110 may be configured to generate a clock signal having a frequency that can be digitally controlled. In particular, in some embodiments, the ring oscillator 112 may be configured to generate a clock signal having a frequency that depends on the amount of current that the ring oscillator 112 receives from the current source unit cells 114. The current source unit cells 114 are coupled to the power supply 130 (generating a voltage that will be referred to as V_{DD}) at one end and couplable to the ring oscillator 112 at the other end. In some embodiments, each of the current source unit cells 114 may be configured to be coupled or decoupled from the ring oscillator 112. When coupled to the ring oscillator 112, each of the current source unit cells 114 may be configured to draw a certain amount of current, Iₒᵤₜ, from the power supply 130 that supply current to the ring oscillator 112. If the number of current source unit cells 114 coupled to the ring oscillator 112 is equal to k, then the total amount of current received by the ring oscillator 112 may be equal to, or approximately equal to or proportional to, or approximately proportional to k×Iₒᵤₜ. As described above, the total amount of current received by the ring oscillator 112 may control the frequency of the clock signal that is generated by the ring oscillator 112, and thus the values of k and Iₒᵤₜ may control the frequency of the clock signal that is generated by the ring oscillator 112.

The bias generation circuitry 104 may be configured to output a bias voltage. The bias generation circuitry 104 may include circuitry configured to generate a bias voltage that is stable despite any changes that may arise in voltage and/or temperature. The control circuitry 108 may be configured to control the bias generation circuitry 104, using one of more digital signals bias_control, to output a particular bias voltage. The output of the bias generation circuitry 104 is coupled to the RC filter 106. The RC filter 106 may include a low-pass filter configured to reduce noise on the bias voltage received from the bias generation circuitry 104 and output a filtered bias voltage V_{bias}. Each of the current source unit cells 114 may receive the filtered bias voltage V_{bias} from the output of the RC filter 106, and the value of V_{bias} may determine the amount of current Iₒᵤₜ that is outputted by each of the current source unit cells 114. As described above, the value of Iₒᵤₜ may control, in part, the frequency of the clock signal that is generated by the ring oscillator 112.

The control circuitry 108 may be configured to control the PLL 102 to cause the DCO 110 to couple a specific number k of the current source unit cells 114 to the ring oscillator 112. The control circuitry 108 is configured to output an input clock signal clk_ref having a particular reference frequency and to provide the input clock signal to the PLL controller 111 of the PLL 102. PLL controller 111 may be configured to generate multi-bit digital control signals based on the frequency of clk_ref and output the control signals to the decoder 122 of the DCO 110. As will be described further below, the PLL controller 111 may use its coarse control circuitry 113 and its fine control circuitry 115 to generate two multi-bit digital control signals based on the frequency of the clk_ref and output the control signals to the decoder 122. In some embodiments, the coarse control circuitry 113 may include frequency detection circuitry and the fine control circuitry 115 may include phase detection circuitry. In some embodiments, coarse_control is modulated by frequency detection circuitry until the frequency detection circuitry finds the optimum frequency band. coarse_control may be fixed when the PLL 102 has completed the frequency locking procedure. In some embodiments, fine_control may be set to half the maximum code (though other values are also possible) when coarse_control is fixed, and then may be modulated by phase detection circuitry. In some embodiments, coarse_control and fine_control may encode information indicating the number k of current source unit cells 114 that should be turned on. In some embodiments, coarse_control may control how many blocks of multiple current source unit cells 114 are turned on, and fine_control may control how many additional current source unit cells 114 may be turned on or off with single current source unit cell resolution. Further description of the coarse control circuitry 113 and the fine control circuitry 115 may be found below.

The decoder 122 may be configured to receive coarse_control and fine_control as inputs and decode them into control signals sw₁, sw₂, sw₃ . . . sw_{N-2}, sw_{N-1}, sw_{N} and/or the inverses of these control signals s̅w̅₁̅, s̅w̅₂̅, s̅w̅₃̅ . . . s̅w̅_̅{̅N̅-̅2̅}̅, s̅w̅_̅{̅N̅-̅1̅}̅, s̅w̅_̅{̅N̅}̅. In some embodiments, these control signals may be single-bit control signals, and in other embodiments, may include more than one bit. The control signals may be provided as inputs to the current source until cells 114. Each of the control signals sw₁, sw₂, sw₃ . . . sw_{N-2}, sw_{N-1}, sw_{N} and/or their inverses s̅w̅₁̅, s̅w̅₂̅, s̅w̅₃̅ . . . s̅w̅_̅{̅N̅-̅2̅}̅, s̅w̅_̅{̅N̅-̅1̅}̅, s̅w̅_̅{̅N̅}̅ may be inputted to a particular one of the current source until cells 114 and may be configured to control whether that particular current source unit cell 114 is coupled to or decoupled from the ring oscillator 112. A particular control signal swₙ may include a bit that is a digital high (e.g., 1) when configured to control a particular current source unit cells 114 to be coupled to the ring oscillator 112 and is a digital low (e.g., 0) when configured to control a particular current source unit cells 114 to be decoupled from the ring oscillator 112 (although the opposite logic may be used in some embodiments). It should be appreciated that, depending on the circuitry within the current source unit cells 114, each of the current source unit cells 114 may receive as inputs a control signal swₙ, its inverse s̅w̅ₙ̅, or both. For example, if a control signal is inputted to the gate of an n-type metal-oxide-semiconductor (nMOS) of a particular current source unit cell 114 to control whether the particular current source unit cell 114 is coupled to or decoupled from the ring oscillator 112, the current source unit cell 114 may receive swₙ. If a control signal is inputted to the gate of a p-type metal-oxide-semiconductor (pMOS) of a particular current source unit cell 114 to control whether the particular current source unit cell 114 is coupled to or decoupled from the ring oscillator 112, the current source unit cell 114 may receive s̅w̅ₙ̅. If control signals are inputted to the gate of an nMOS and a pMOS of a particular current source unit cell 114 to control whether the particular current source unit cell 114 is coupled to or decoupled from the ring oscillator 112, the current source unit cell 114 may receive both swₙ and s̅w̅ₙ̅.

As a non-limiting example, consider that each of the current source unit cells 114 outputs current to the ring oscillator 112 when receiving a control signal swₙ that is digital high. For given values of coarse_control and fine_control, the decoder 122 may output control signals such that sw₁, sw₂, sw₃ . . . swₙ are digital high, thereby causing the current source until cells 114 receiving these control signals to output current to the ring oscillator 112, and the decoder 122 may output control signals such that swₙ₊₁, swₙ₊₂, swₙ₊₃ . . . sw_{N} are digital low, thereby causing the current source until cells 114 receiving these control signals to not output current to the ring oscillator 112. In other words, the values coarse_control and fine_control may control k, namely how many current source until cells 114 are supplying current to the ring oscillator 112. As described above, the value of k may determine, in part, the frequency of the clock signal that is generated by the ring oscillator 112. Thus, because the control circuitry 108 may control k and Iₒᵤₜ, as described above, the control circuitry 108 may control the frequency of the clock signal that is generated by the ring oscillator 112.

In some embodiments, the PLL 102 may use fast switching techniques. For example, the PLL 102 may include transceiver circuitry that allows the PLL 102 to power down and conserve power when the ultrasound device is not generating data, and power up to full operation within an acceptably fast period of time (e.g., within 1 microsecond) when the ultrasound device begins to generate data again. For further description of fast techniques, see Wei, Da, et al., “A 10-Gb/s/ch, 0.6-pJ/bit/mm Power Scalable Rapid-ON AND/OR OFF Transceiver for On-Chip Energy Proportional Interconnects,” I9 Journal of Solid-State Circuits 53.3 (2018): 873-883. When each of the current source unit cells 114 switches on and off using such a fast switching technique, the current source unit cells 114 may cause a large disturbance that may couple to the node V_{bias}. In particular, when switching on or off, each of the current source unit cells 114 may experience a voltage transition (e.g., a voltage spike and the associated settling time) at an internal node that may couple to the node V_{bias}. The combined effect of all voltage transitions at all the current source unit cells 114 coupling to the node V_{bias} may cause a large disturbance in the voltage at V_{bias}. This large disturbance may cause an increase in the settling time of V_{bias}. If the voltage V_{bias} does not settle fast, then the frequency of the output signal from the DCO 110 may not settle in an acceptably short time period. This may negatively affect the performance of the ultrasound device. For example, a frequency of the output signal from the DCO 110 not settling in an acceptably short time period may result in bit errors. Bit errors, in turn, may lead to blurry images or, in some instances, to image frame loss.

The inventors have realized that using two types of current source unit cells 114 may help to reduce the disturbance caused by fast switching of the current source unit cells 114. In some embodiments, one type of current source unit cell includes a drain switch 114a and another type of current source unit cell includes a source switch 114b, although in other embodiments the types of current source unit cell may differ from one another in other respects. The current source unit cells with drain switch 114a may have a different circuit topology than the current source unit cells with source switch 114b. In particular, the topologies may be such that fast switching of the current source unit cells with drain switch 114a causes a voltage transition that proceeds in one voltage direction (e.g., the voltage increases) while fast switching of the current source unit cells with source switch 114a causes a voltage transition that proceeds in the opposite voltage direction (e.g., if the drain switch causes the voltage to increases, the source switch may cause the voltage to decrease, or vice versa). Thus, the voltage transitions of multiple current source unit cells 114 of the two types may cancel each other out to a certain degree. The total disturbance that may couple to the node V_{bias} may be therefore be less than if all the current source unit cells 114 were of a single type and all the voltage transitions proceeded in the same direction.

Consider the following non-limiting example. As illustrated in FIG. 1, the current source unit cells 114 are coupled to a power supply 130 generating a voltage that will be referred to as V_{DD}. The voltage of the output nodes of the current source unit cells 114, which are couplable to the ring oscillator 112, will be referred to as Vₒᵤₜ. When the current source unit cells with drain switch 114a turn on, nodes internal to the current source unit cells with drain switch 114a may transition from V_{DD} to Vₒᵤₜ, and the transition at these nodes may couple to the node V_{bias}. When the current source unit cells with source switch 114b turn on, nodes internal to the current source unit cells with source switch 114b may transition from Vₒᵤₜ to V_{DD}, and the transition at these nodes may couple to the node V_{bias}. It should be appreciated that these two transitions proceed in opposite voltage directions and thus may cancel each other out to a certain degree.

It should be appreciated that, when turning on a certain number (e.g., k) of the current source unit cells 114 as described above, this may include turning a first number of the current source unit cells with drain switch 114a and a second number of the current source unit cells with source switch 114b, and the first and second numbers may be different in some embodiments.

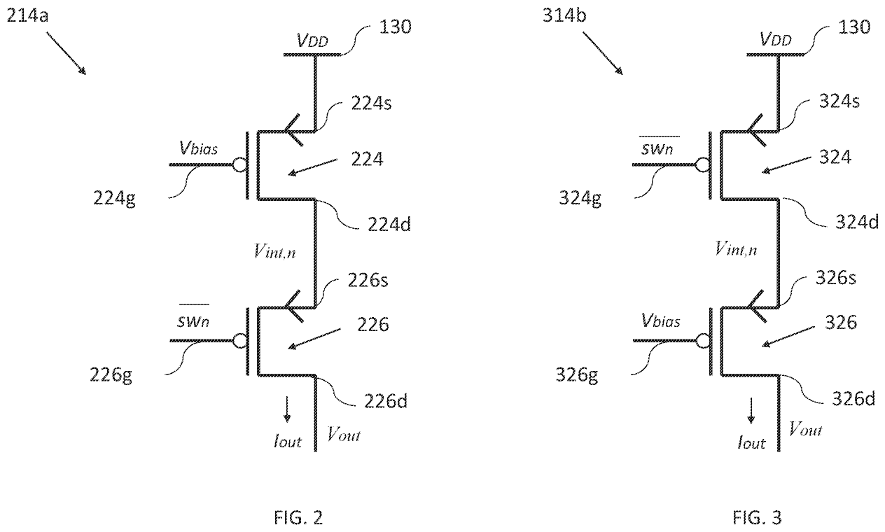

FIG. 2 illustrates a circuit diagram of a current source unit cell with drain switch 214a, in accordance with certain embodiments described herein. In some embodiments, some or all of the current source unit cells with drain switch 114a in FIG. 1 may have the topology of the current source unit cell with drain switch 214a. The current source unit cell with drain switch 214a includes a first p-type metal-oxide-semiconductor (pMOS) transistor 224 and a second pMOS transistor 226. The first pMOS transistor 224 includes a gate terminal 224g, a drain terminal 224d, and a source terminal 224s. The second pMOS transistor 226 includes a gate terminal 226g, a drain terminal 226d, and a source terminal 226s. The gate terminal 224g of the first pMOS transistor 224 is coupled to the node V_{bias} (i.e., the output of the RC filter 106). The source terminal 224s of the first pMOS transistor 224 is coupled to the power supply 130. The drain terminal 224d of the first pMOS transistor 224 is coupled to the source terminal 226s of the second pMOS transistor 226 at an internal node (referred to as V_{int,n}) not shared with other current source unit cells 114. The gate terminal 226g of the second pMOS transistor 226 is coupled to a signal s̅w̅ₙ̅ (i.e., one of the outputs of the decoder 122). The drain terminal 226d of the second pMOS transistor 226 is couplable to the ring oscillator 112 at a node (referred to as Vₒᵤₜ) that is shared by other current source unit cells 114. The first pMOS transistor 224 functions as a current source, generating the current Iₒᵤₜ having a value depending on the value of V_{bias}. The second pMOS transistor 226 functions as a switch, enabling (when s̅w̅ₙ̅ is low) or disabling (when s̅w̅ₙ̅ is high) the flow of the current Iₒᵤₜ from the first pMOS transistor 224 to the ring oscillator 112. It should be appreciated that the current source unit cell with drain switch 214a is referred to as having a “drain switch” because the second pMOS transistor 226, which functions as a switch, is coupled to the drain 224d of the first pMOS transistor 224, which functions as a current source.

Refer to the voltage at the power supply 130 node as V_{DD}, the voltage at the drain terminal 226d of the second pMOS transistor 226 which is couplable to the ring oscillator 112 as Vₒᵤₜ, and the voltage at the internal node to which the drain terminal 224d of the first pMOS transistor 224 and the source terminal 226s of the second pMOS transistor 226 are coupled as V_{int,n}. When the second pMOS transistor 226d, which functions as a switch, turns on, the voltage V_{int,n} may transition from V_{DD} to Vₒᵤₜ. When the second pMOS transistor 226d, which functions as a switch, turns off, the voltage V_{int,n} may transition from Vₒᵤₜ to V_{DD}.

FIG. 3 illustrates a circuit diagram of a current source unit cell with source switch 314b, in accordance with certain embodiments described herein. In some embodiments, some or all of the current source unit cells with source switch 114b in FIG. 1 may have the topology of the current source unit cell with source switch 314b. The current source unit cell with source switch 314b includes a first p-type metal-oxide-semiconductor (pMOS) transistor 324 and a second pMOS transistor 326. The first pMOS transistor 324 includes a gate terminal 324g, a drain terminal 324d, and a source terminal 324s. The second pMOS transistor 326 includes a gate terminal 326g, a drain terminal 326d, and a source terminal 326s. The gate terminal 324g of the first pMOS transistor 324 is coupled to a signal s̅w̅ₙ̅ (i.e., one of the outputs of the decoder 122). The source terminal 324s of the first pMOS transistor 324 is coupled to the power supply 130. The drain terminal 324d of the first pMOS transistor 324 is coupled to the source terminal 326s of the second pMOS transistor 326 at an internal node (referred to as V_{int,n}) not shared with other current source unit cells 114. The gate terminal 326g of the second pMOS transistor 326 is coupled to the node V_{bias} (i.e., the output of the RC filter 106). The drain terminal 326d of the second pMOS transistor 326 is couplable to the ring oscillator 112 at a node (referred to as Vₒᵤₜ) that is shared by other current source unit cells 114. The second pMOS transistor 326 functions as a current source, generating the current Iₒᵤₜ having a value depending on the value of V_{bias}. The first pMOS transistor 324 functions as a switch, enabling (when s̅w̅ₙ̅ is low) or disabling (when s̅w̅ₙ̅ is high) the flow of the current Iₒᵤₜ from the second pMOS transistor 326 to the ring oscillator 112. It should be appreciated that the current source unit cell with source switch 314b is referred to as having a “source switch” because the first pMOS transistor 324, which functions as a switch, is coupled to the source 324s of the second pMOS transistor 326, which functions as a current source.

Refer to the voltage at the power supply 130 node as V_{DD}, the voltage at the drain terminal 326d of the second pMOS transistor 326 which is couplable to the ring oscillator 112 as Vₒᵤₜ, and the voltage at the internal node to which the drain terminal 324d of the first pMOS transistor 324 and the source terminal 326s of the second pMOS transistor 326 are coupled as V_{int,n}. When the second pMOS transistor 326d, which functions as a switch, turns on, the voltage V_{int,n} may transition from Vₒᵤₜ to V_{DD}. When the second pMOS transistor 226d, which functions as a switch, turns off, the voltage V_{int,n} may transition from V_{DD} to Vₒᵤₜ.

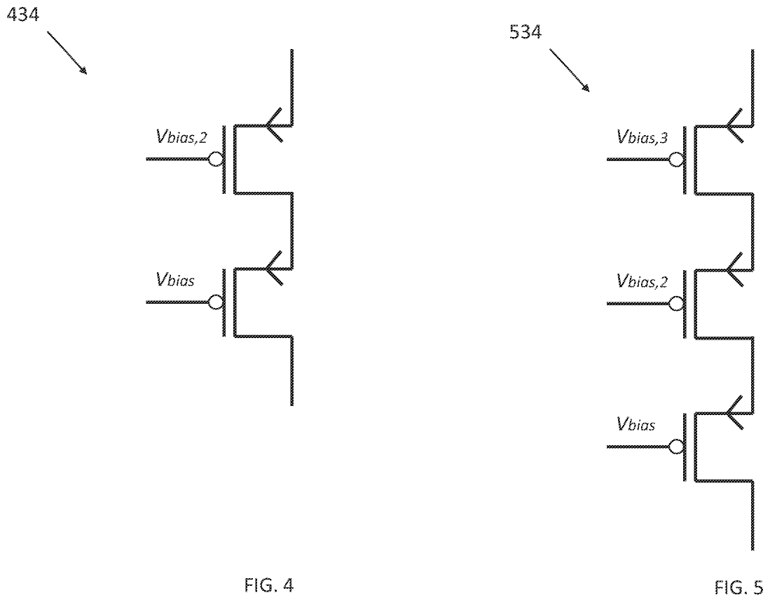

FIG. 4 illustrates a circuit diagram of a current source 434, in accordance with certain embodiments described herein. The current source 434 may be considered to be a cascode current source. The current source 434 includes two pMOS transistors. The gate of one of the pMOS transistors is coupled to the node V_{bias}. The gate of the other pMOS transistor is coupled to another bias voltage V_{bias,2}. The current source 434 may be used instead of a pMOS transistor functioning as a current source in any of the current source unit cells described above with reference to FIGS. 2-3. For example, a current source unit cell with drain switch may be formed by using the current source 434 instead of the first pMOS transistor 224 in the current source unit cell with drain switch 214a. As another example, a current source unit cell with source switch may be formed by using the current source 434 instead of the second pMOS transistor 326 in the current source unit cell with source switch 314b.

FIG. 5 illustrates a circuit diagram of a current source 534, in accordance with certain embodiments described herein. The current source 534 may be considered to be a double-cascode current source. The current source 534 includes three pMOS transistors. The gate of one of the pMOS transistors is coupled to the node V_{bias}. The gates of the other two pMOS transistors are coupled to two other bias voltages, V_{bias,2} and V_{bias,3}, respectively. The current source 534 may be used instead of a pMOS transistor functioning as a current source in any of the current source unit cells described herein. For example, a current source unit cell with drain switch may be formed by using the current source 534 instead of the first pMOS transistor 224 in the current source unit cell with drain switch 214a. As another example, a current source unit cell with source switch may be formed by using the current source 534 instead of the second pMOS transistor 326 in the current source unit cell with source switch 314b.

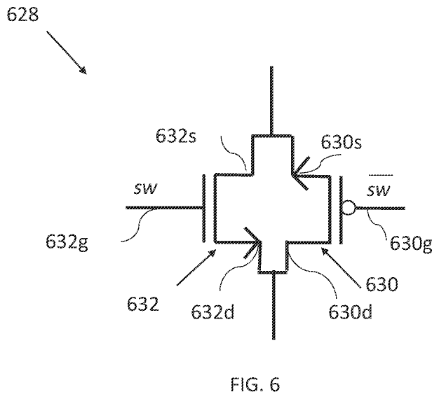

FIG. 6 illustrates a circuit diagram of a transmission gate 628, in accordance with certain embodiments described herein. The transmission gate 628 includes a pMOS transistor 630 and an nMOS transistor 632. The pMOS transistor 630 includes a gate terminal 630g, a drain terminal 630d, and a source terminal 630s. The nMOS transistor 632 includes a gate terminal 632g, a drain terminal 632d, and a source terminal 632s. The gate terminal 632g of the nMOS transistor 632 is coupled to a signal swₙ (i.e., one of the outputs of the decoder 122) and the gate terminal 630g of the pMOS transistor 630 is coupled to s̅w̅ₙ̅, the inverse of the signal swn. The drain terminal 630d of the pMOS transistor 630 is coupled to the drain terminal 632d of the nMOS transistor 632. The source terminal 630s of the pMOS transistor 630 is coupled to the source terminal 632s of the pMOS transistor 632.

The transmission gate 628 may be used instead of a transistor functioning as a switch in any of the current source unit cells described with reference to FIGS. 2-5. For example, a current source unit cell with drain switch 114a may be formed by using the transmission gate 628 instead of the second pMOS transistor 226 in the current source unit cell with drain switch 214a (whether with or without substitution of the current source 434 or the current source 534 for the first pMOS transistor 224). As another example, a current source unit cell with source switch 114b may be formed by using the transmission gate 628 instead of the first pMOS transistor 324 in the current source unit cell with source switch 314b (whether with or without substitution of the current source 434 or the current source 534 for the second pMOS transistor 326).

Referring back to FIG. 1, the current source unit cells with drain switch 114a may have the topology of the current source unit cell with drain switch 214a. In some embodiments, the topology of the current source unit cell with drain switch 214a may be substituted with the current source 434. In some embodiments, the topology of the current source unit cell with drain switch 214a may be substituted with the current source 534. In some embodiments, the topology of the current source unit cell with drain switch 214a may be substituted with the transmission gate 628 In some embodiments, the topology of the current source unit cell with drain switch 214a may be substituted with the current source 434 and the transmission gate 628, or the topology of the current source unit cell with drain switch 214a may substituted with the current source 534 and the transmission gate 628. In some embodiments, all the current source unit cells with drain switch 114a have the same topology. Alternatively, some of the current source unit cells with drain switch 114a may have one of the topologies described above, others may have others of these topologies, etc. The current source unit cells with source switch 114b may have the topology of the current source unit cell with source switch 314b. In some embodiments, the topology of the current source unit cell with source switch 314b may be substituted with the current source 434. In some embodiments, the topology of the current source unit cell with source switch 314b may be substituted with the current source 534. In some embodiments, the topology of the current source unit cell with source switch 314b may be substituted with the transmission gate 628. In some embodiments, the topology of the current source unit cell with source switch 314b may be substituted with the current source 434 and the transmission gate 628, or the topology of the current source unit cell with source switch 314b may be substituted with the current source 534 and the transmission gate 628. In some embodiments, all the current source unit cells with source switch 114b have the same topology. Alternatively, some of the current source unit cells with source switch 114b may have one of the topologies described above, others may have others of these topologies, etc.

It should be appreciated from the above description that, in any of the topologies for the current source unit cells with drain switch 114a described above, when the switch of the current source unit cell turns on to allow current to flow from the current source, the voltage of the internal node V_{int,n} may transition from V_{DD} to Vₒᵤₜ. When the switch of the current source unit cell turns off to prevent current from flowing from the current source, the voltage of the internal node V_{int,n} may transition from Vₒᵤₜ to V_{DD} . By contrast, in any of the topologies for the current source unit cells with source switch 114b described above, when the switch of the current source unit cell turns on to allow current to flow from the current source, the voltage of the internal node V_{int,n} may transition from Vₒᵤₜ to V_{DD} . When the switch of the current source unit cell turns off to prevent current from flowing from the current source, the voltage of the internal node V_{int,n} may transition from V_{DD} to Vₒᵤₜ. Thus, the voltage transition at the internal node V_{int,n} when switching on may proceed in the opposite direction for current source unit cells with drain switch compared with that with source switch, and the voltage transition at the internal node V_{int,n} when switching on may proceed in the opposite direction for current source unit cells with drain switch compared with that with source switch, Thus, if one or more current source unit cells with drain switch and one or more current source unit cells with source switch on or off together, the voltage transitions at the internal nodes V_{int,n} of each of the current source unit cells of the first and second types may proceed in opposite voltage directions and may cancel each other out to a certain degree. The total disturbance that may couple to the node V_{bias} from the internal nodes V_{int,n} may therefore be less than if all the current source unit cells were of a single type and all the voltage transitions proceeded in the same direction.

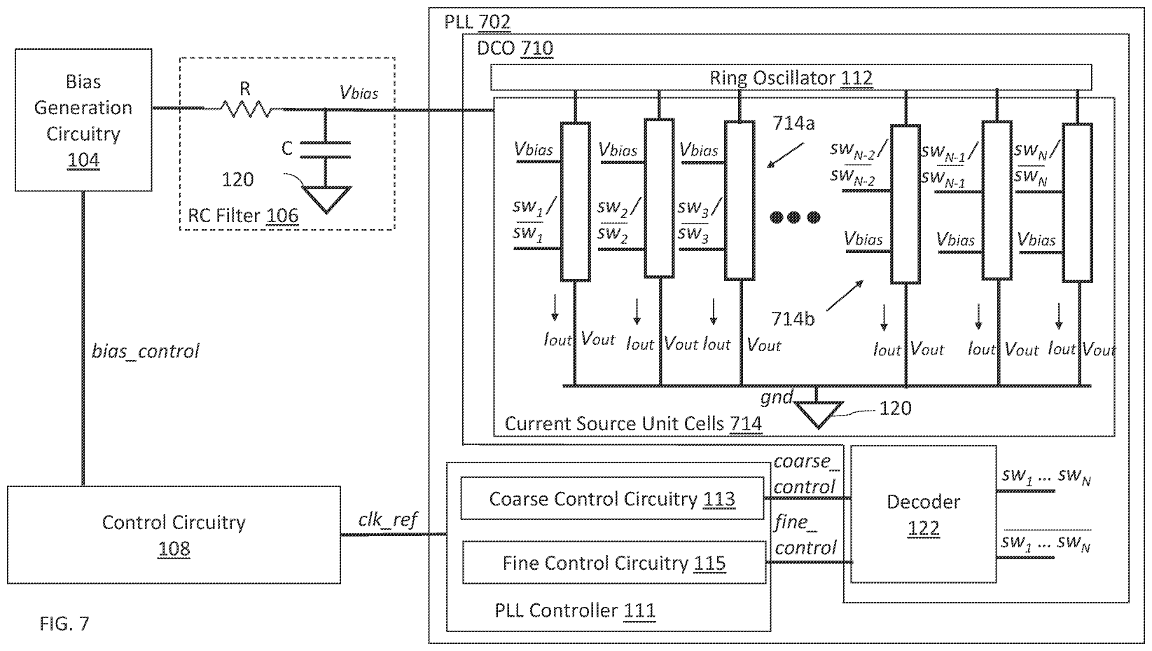

FIG. 7 illustrates a schematic block diagram of circuitry in an ultrasound device, in accordance with certain embodiments described herein. The circuitry in FIG. 7 is is similar in some respects to the circuitry in FIG. 1 except that the circuitry in FIG. 7 includes the PLL 702, which includes the DCO 710, instead of the PLL 102 and the DCO 110. The DCO 710 includes current source unit cells 714, which include current source unit cells with drain switch 714a and current source unit cells with source switch 714b. The current source unit cells 714 are coupled to ground 120 at one end and couplable to the ring oscillator 112 at the other end. In some embodiments, each of the current source unit cells 714 may be configured to be coupled or decoupled from the ring oscillator 112. In contrast to the current source unit cells 114, when coupled to the ring oscillator 112, each of the current source unit cells 114 may be configured to sink a certain amount of current, Iₒᵤₜ, from the ring oscillator 114 and supply that current to ground 120.

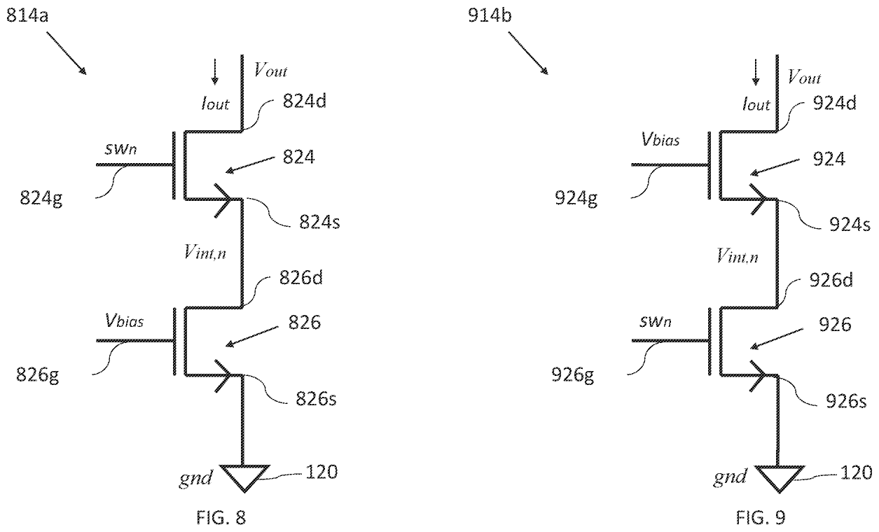

FIG. 8 illustrates a circuit diagram of a current source unit cell with drain switch 814a, in accordance with certain embodiments described herein. In some embodiments, some or all of the current source unit cells with drain switch 714a in FIG. 7 may have the topology of the current source unit cell with drain switch 814a. The current source unit cell with drain switch 814a includes a first n-type metal-oxide-semiconductor (nMOS) transistor 824 and a second nMOS transistor 826. The first nMOS transistor 824 includes a gate terminal 824g, a drain terminal 824d, and a source terminal 824s. The second nMOS transistor 826 includes a gate terminal 826g, a drain terminal 826d, and a source terminal 826s. The gate terminal 824g of the first nMOS transistor 824 is coupled to a signal swₙ (i.e., one of the outputs of the decoder 122). The drain terminal 824d of the first nMOS transistor 824 is couplable to the ring oscillator 112 at a node (referred to as Vₒᵤₜ) that is shared by other current source unit cells 114. The source terminal 824s of the first nMOS transistor 824 is coupled to the drain terminal 826d of the second nMOS transistor 826 at an internal node (referred to as V_{int,n}) not shared with other current source unit cells 714. The gate terminal 826g of the second nMOS transistor 826 is coupled to a signal the node V_{bias} (i.e., the output of the RC filter 106). The source terminal 826s of the second nMOS transistor 826 is coupled to ground 120. The second nMOS transistor 826 functions as a current source, generating the current Iₒᵤₜ having a value depending on the value of V_{bias}. The first nMOS transistor 824 functions as a switch, enabling (when swₙ is high) or disabling (when swₙ is low) the flow of the current Iₒᵤₜ from the second nMOS transistor 826 to the ring oscillator 112. It should be appreciated that the current source unit cell with drain switch 814a is referred to as having a “drain switch” because the first nMOS transistor 824, which functions as a switch, is coupled to the drain 824d of the second nMOS transistor 826, which functions as a current source.

Refer to the voltage at ground 120 as gnd, the voltage at the source terminal 826s of the second nMOS transistor 826 which is couplable to the ring oscillator 112 as Vₒᵤₜ, and the voltage at the internal node to which the source terminal 824s of the first nMOS transistor 824 and the drain terminal 826d of the second nMOS transistor 826 are coupled as V_{int,n}. When the first nMOS transistor 824, which functions as a switch, turns on, the voltage V_{int,n} may transition from gnd to Vₒᵤₜ. When the first nMOS transistor 824, which functions as a switch, turns off, the voltage V_{int,n} may transition from Vₒᵤₜ to gnd.

FIG. 9 illustrates a circuit diagram of a current source unit cell with source switch 914b, in accordance with certain embodiments described herein. In some embodiments, some or all of the current source unit cells with source switch 714b in FIG. 7 may have the topology of the current source unit cell with source switch 914b. The current source unit cell with source switch 914b includes a first n-type metal-oxide-semiconductor (nMOS) transistor 924 and a second nMOS transistor 926. The first nMOS transistor 924 includes a gate terminal 924g, a drain terminal 924d, and a source terminal 924s. The second nMOS transistor 926 includes a gate terminal 926g, a drain terminal 926d, and a source terminal 926s. The gate terminal 924g of the first nMOS transistor 924 is coupled to the node V_{bias} (i.e., the output of the RC filter 106). The drain terminal 924d of the first nMOS transistor 924 is couplable to the ring oscillator 112 at a node (referred to as Vₒᵤₜ) that is shared by other current source unit cells 114. The source terminal 924s of the first nMOS transistor 924 is coupled to the drain terminal 926d of the second nMOS transistor 926 at an internal node (referred to as V_{int,n}) not shared with other current source unit cells 714. The gate terminal 926g of the second nMOS transistor 926 is coupled to a signal swₙ (i.e., one of the outputs of the decoder 122). The source terminal 926s of the second nMOS transistor 926 is coupled to ground 120. The first nMOS transistor 924 functions as a current source, generating the current Iₒᵤₜ having a value depending on the value of V_{bias}. The second nMOS transistor 926 functions as a switch, enabling (when swₙ is high) or disabling (when swₙ is low) the flow of the current Iₒᵤₜ from the first nMOS transistor 924 to the ring oscillator 112. It should be appreciated that the current source unit cell with source switch 914b is referred to as having a “source switch” because the second nMOS transistor 926, which functions as a switch, is coupled to the source 924s of the first nMOS transistor 924, which functions as a current source.

Refer to the voltage at ground 120 as gnd, the voltage at the source terminal 926s of the second nMOS transistor 926 which is couplable to the ring oscillator 112 as Vₒᵤₜ, and the voltage at the internal node to which the source terminal 924s of the first nMOS transistor 924 and the drain terminal 926d of the second nMOS transistor 926 are coupled as V_{int,n}. When the second nMOS transistor 926, which functions as a switch, turns on, the voltage V_{int,n} may transition from Vₒᵤₜ to gnd. When the second nMOS transistor 926d, which functions as a switch, turns off, the voltage V_{int,n} may transition from gnd to Vₒᵤₜ.

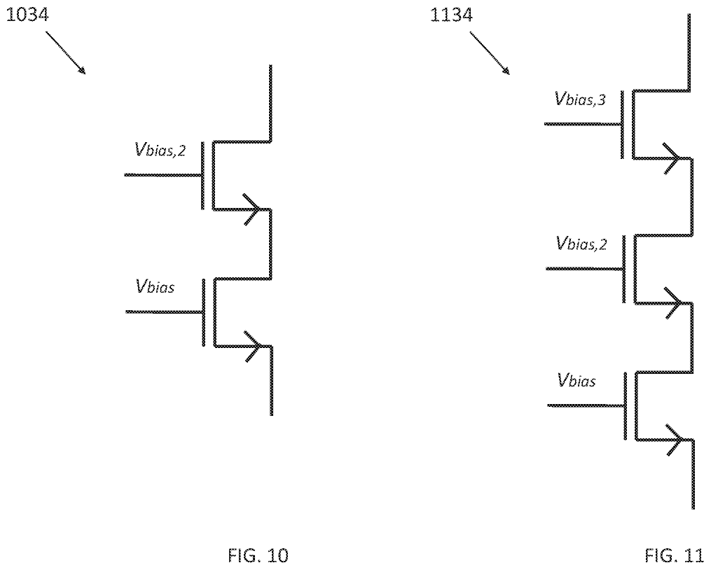

FIG. 10 illustrates a circuit diagram of a current source 1034, in accordance with certain embodiments described herein. The current source 1034 may be considered to be a cascode current source. The current source 1034 includes two nMOS transistors. The gate of one of the nMOS transistors is coupled to the node V_{bias}. The gate of the other nMOS transistor is coupled to another bias voltage V_{bias,2}. The current source 1034 may be used instead of a nMOS transistor functioning as a current source in any of the current source unit cells described herein. For example, a current source unit cell with source switch may be formed by using the current source 1034 instead of the second pMOS transistor 826 in the current source unit cell with source switch 814a (whether with or without substitution of the transmission gate 628). As another example, a current source unit cell with drain switch may be formed by using the current source 1034 instead of the first nMOS transistor 924 in the current source unit cell with drain switch 914b (whether with or without substitution of the transmission gate 628).

FIG. 11 illustrates a circuit diagram of a current source 1134, in accordance with certain embodiments described herein. The current source 1134 may be considered to be a double-cascode current source. The current source 1134 includes three nMOS transistors. The gate of one of the nMOS transistors is coupled to the node V_{bias}. The gates of the other two nMOS transistors are coupled to two other bias voltages, V_{bias,2} and V_{bias,3}, respectively. The current source 1134 may be used instead of a nMOS transistor functioning as a current source in any of the current source unit cells described herein. For example, a current source unit cell with source switch may be formed by using the current source 1134 instead of the second pMOS transistor 826 in the current source unit cell with source switch 814a. As another example, a current source unit cell with drain switch may be formed by using the current source 1134 instead of the first nMOS transistor 924 in the current source unit cell with drain switch 914b.

Referring back to FIG. 6, the transmission gate 628 may be used instead of a transistor functioning as a switch in any of the current source unit cells described with reference to FIGS. 8-11. For example, a current source unit cell with drain switch 714a may be formed by using the transmission gate 628 instead of the first nMOS transistor 824 in the current source unit cell with source switch 814a (whether with or without substitution of the current source 1034 or the current source 1134 for the first nMOS transistor 824). As another example, a current source unit cell with drain switch may be formed by using the transmission gate 628 instead of the second nMOS transistor 926 in the current source unit cell with drain switch 914b (whether with or without substitution of the current source 1034 or the current source 1134 for the second nMOS transistor 926).

Referring back to FIG. 7, the current source unit cells with drain switch 714a may have the topology of the current source unit cell with drain switch 814a, the topology of the current source unit cell with drain switch 814a being substituted with the current source 1034. In some embodiments, the topology of the current source unit cell with drain switch 814a may be substituted with the current source 1134. In some embodiments, the topology of the current source unit cell with drain switch 814a may be substituted with the transmission gate 628. In some embodiments, the topology of the current source unit cell with drain switch 814a may be substituted with the current source 1034 and the transmission gate 628, or the topology of the current source unit cell with drain switch 814a may be substituted with the current source 1134 and the transmission gate 628. In some embodiments, all the current source unit cells with drain switch 714a have the same topology. Alternatively, some of the current source unit cells with drain switch 714a may have one of the topologies described above, others may have others of these topologies, etc. The current source unit cells with source switch 714b may have the topology of the current source unit cell with source switch 914b. In some embodiments, the topology of the current source unit cell with source switch 914b may be substituted with the current source 1034. In some embodiments, the topology of the current source unit cell with source switch 914b may be substituted with the current source 1134. In some embodiments, the topology of the current source unit cell with source switch 914b may be substituted with the transmission gate 628. In some embodiments, the topology of the current source unit cell with source switch 914b may be substituted with the current source 1134 and the transmission gate 628, or the topology of the current source unit cell with source switch 914b may be substituted with the current source 1134 and the transmission gate 628. In some embodiments, all the current source unit cells with source switch 714b have the same topology. Alternatively, some of the current source unit cells with source switch 714b may have one of the topologies described above, others may have others of these topologies, etc.

It should be appreciated from the above description that, in any of the topologies for the current source unit cells with drain switch 714a described above, when the switch of the current source unit cell turns on to allow current to flow from the current source, the voltage of the internal node V_{int,n} may transition from gnd to Vₒᵤₜ. When the switch of the current source unit cell turns off to prevent current from flowing from the current source, the voltage of the internal node V_{int,n} may transition from Vₒᵤₜ to gnd. By contrast, in any of the topologies for the current source unit cells with source switch 714b described above, when the switch of the current source unit cell turns on to allow current to flow from the current source, the voltage of the internal node V_{int,n} may transition from Vₒᵤₜ to gnd. When the switch of the current source unit cell turns off to prevent current from flowing from the current source, the voltage of the internal node V_{int,n} may transition from gnd to Vₒᵤₜ. Thus, the voltage transition at the internal node V_{int,n} when switching on may proceed in the opposite direction for current source unit cells with drain switch compared with that with source switch, and the voltage transition at the internal node V_{int,n} when switching on may proceed in the opposite direction for current source unit cells with drain switch compared with that with source switch, Thus, if one or more current source unit cells with drain switch and one or more current source unit cells with source switch on or off together, the voltage transitions at the internal nodes V_{int,n} of each of the current source unit cells of the first and second types may proceed in opposite voltage directions and may cancel each other out to a certain degree. The total disturbance that may couple to the node V_{bias} from the internal nodes V_{int,n} may be therefore be less than if all the current source unit cells were of a single type and all the voltage transitions proceeded in the same direction.

While the above description describes various example topologies of current source unit cells with drain switch and current source unit cells with source switch, these topologies are non-limiting. Generally, a current source unit cell with drain switch or the second type may be formed from a switch and a current source. The switch may be controlled by a signal swₙ (i.e., one of the outputs of the decoder 122) or its inverse s̅w̅ₙ̅. For example, the switch may be a single transistor or a transmission gate. The current source may include at least one transistor having a gate coupled to V_{bias}, and have two terminals. A current source unit cell with drain switch may be formed by coupling the switch to one terminal of the current source, and a current source unit cell with source switch may be formed by coupling the switch to the other terminal of the current source. In some embodiments, the current source may be a single transistor, and the current source unit cell with drain switch may have a switch coupled to the drain terminal of the single transistor, and the current source unit cell with source switch may have a switch coupled to the source terminal of the single transistor. In some embodiments, the current source may be a cascode current source including multiple transistors, and the current source unit cell with drain switch may have a switch coupled to the drain terminal of one of the transistors, and the current source unit cell with source switch may have a switch coupled to the source terminal of one of the transistors.

More generally, a current source unit cell with drain switch may have one circuit topology and a current source unit cell with source switch may have a different circuit topology, such that when the current source unit cell with drain switch switches on to allow current to flow, the voltage transition at one of its internal nodes proceeds in an opposite voltage direction than when the current source unit cell with source switch switches on to allow current flow, and when the current source unit cell with drain switch switches off to prevent current from flowing, the voltage transition at one of its internal nodes proceeds in an opposite voltage direction than when the current source unit cell with source switch switches off to prevent current from flowing.

Described above are examples of current source unit cells implemented using MOS transistors. However, not all embodiments are limited to these particular types of transistors. Other transistors may be used additionally or alternatively, including for example bipolar junction transistors (BJTs) and junction field effect transistors (JFETs), among other examples. For example, in the implementations described above, pMOS transistors may be replaced by p-n-p BJTs and nMOS transistors may be replaced by n-p-n BJTs. Accordingly, as used herein, the term “gate” may be used to identify either the gate of a field effect transistor or the base of a bipolar transistor; the term “drain” may be used to identify either the drain of a field effect transistor or the collector of a bipolar transistor; and the term “source” may be used to identify either the source of a field effect transistor or the emitter of a bipolar transistor.

The following description will analyze the voltage disturbance that may occur in the examples of the current source unit cell with drain switch 214a and the current source unit cell with source switch 314b, as well as optimization of patterns for turning on and off these example current source unit cells. Similar analysis may be used for any other current source unit cells described herein.

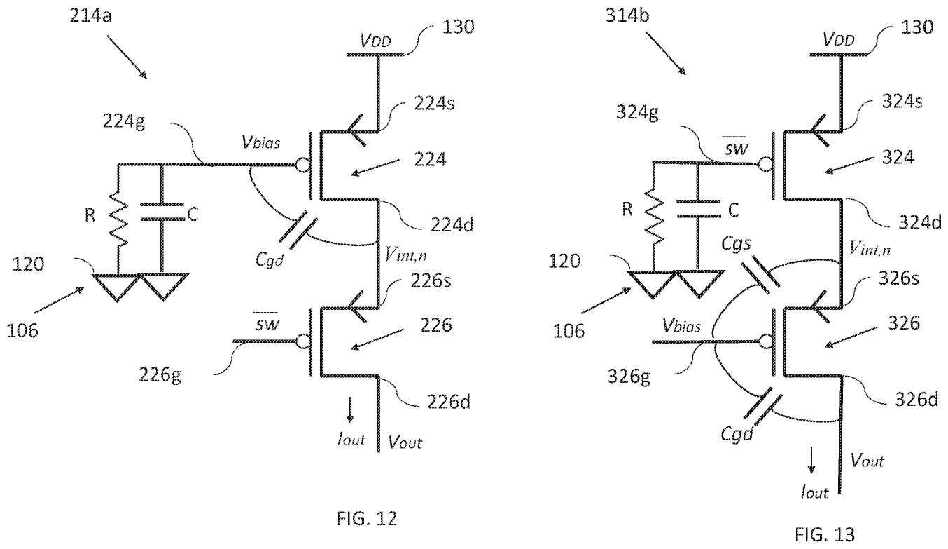

FIG. 12 illustrates the current source unit cell with drain switch 214a with the addition of a parasitic capacitance, in accordance with certain embodiments described herein. In particular, FIG. 12 illustrates a parasitic capacitance Cgd between the gate 224g and the drain 224d of the first pMOS transistor 224. FIG. 12 further illustrates a small-signal equivalent for the RC filter 106 in which V_{bias} is assumed to be an AC ground, such that the resistor R and the capacitor C are in parallel to each other. The combined impedance ZL of the RC filter 106 may then be (R∥(1/sC)), where s represents a complex frequency parameter. In some embodiments, C may be large compared to R, such that ZL is approximately equal to 0.

V_{int,n} may couple to V_{bias} through Cgd. In particular,

$${V}_{bias} = \frac{{Z}_{L}}{{Z}_{L} + \frac{1}{{sC}_{gd}}} {V}_{int} .$$

Assume sC_{gd}Z_{L}<<1, then V_{bias}=sC_{gd}Z_{L}Vᵢₙₜ.

V_{int,n} may couple to V_{bias} through Cgd. In particular,

$${V}_{bias} = \frac{{Z}_{L}}{{Z}_{L} + \frac{1}{{sC}_{gd}}} {V}_{int} .$$

Assume sC_{gd}Z_{L}<<, then V_{bias}=sC_{gd}Z_{L}Vᵢₙₜ. Assume as a particular example that Cgd=2.1 fF and the voltage at V_{int,n} decreases by 196 mV when the current source unit cell with drain switch 214a turns on (e.g., the second pMOS transistor 226, functioning as a switch, begins to allow current to flow from the first pMOS transistor 224, functioning as a current source). (This value may be calculated using simulation.) Then, based on the above equation, the voltage at V_{bias} may decrease by 411.6×10⁻¹⁵×sZL mV due to the turning on of one current source until cell with drain switch 214a.

FIG. 13 illustrates the current source unit cell with source switch 314b with the addition of parasitic capacitances, in accordance with certain embodiments described herein. In particular, FIG. 13 illustrates a parasitic capacitance Cgd between the gate 326g and the drain 326d of the second pMOS transistor 326 and a parasitic capacitance Cgs between the gate 326g and the source 326s of the second pMOS transistor 326. FIG. 13 further illustrates the small-signal equivalent for the RC filter 106 described in more detail above with reference to FIG. 12.

V_{int,n} and Vₒᵤₜ may couple to V_{bias} through Cgs and Cgd, respectively. In particular,

$${V}_{bias} = \frac{{Z}_{L}}{{Z}_{L} + \frac{1}{{sC}_{gd}}} {V}_{int} + \frac{{Z}_{L}}{{Z}_{L} + \frac{1}{{sC}_{gd}}} {V}_{IOUT} .$$

Assume sC_{gd}Z_{L}<<1 and sC_{gs}Z_{L}<<1, then V_{bias}=sC_{gs}Z_{L}Vᵢₙₜ+sC_{gd}Z_{L}V_{IOUT}. Assume as a particular example that Cgd=2.0 fF, Cgs=0.65 fF, the voltage at V_{int,n} increases by 348 mV when the current source unit cell with source switch 314b turns on (e.g., the first pMOS transistor 324, functioning as a switch, begins to allow current to flow from the second pMOS transistor 326, functioning as a current source), and the voltage at Vₒᵤₜ increases by 514 mV when the current source unit cell with source switch 314b turns on. (This value may be calculated using simulation.) Then, based on the above equation, the voltage at V_{bias} may increase by 1254.2×10⁻¹⁵×sZL mV due to the turning on of one current source until cell with drain switch 214a.

It should thus be appreciated that in the example above, the current source unit cell 314b, when turning on, causes an increase in voltage at V_{bias} that is approximately 3 times as large as the decrease in voltage at V_{bias} caused by the current source unit cell 214a turning on. In other words, in this example, the ratio between the decrease in voltage at V_{bias} caused by the current source unit cell with drain switch 214a turning on to the increase in voltage at V_{bias} caused by the current source unit cell with source switch 314b turning on is approximately 1:3. Generally, the ratio between the decrease in voltage at V_{bias} caused by the current source unit cell with drain switch 214a turning on to the increase in voltage at V_{bias} caused by the current source unit cell with source switch 314b turning on may be C_{gd}V_{int,D}:C_{gs}V_{int,S}+C_{gd}V_{out,S}. In this equation, V_{int,D} is the decrease in voltage at V_{int,n} in the current source unit cell with drain switch 214a due the current source unit cell with drain switch 214a turning on, V_{int,S} is the increase in voltage at V_{int,n} in the current source unit cell with source switch 314b due the current source unit cell with source switch 314b turning on, and V_{out,S} is the increase in voltage at Vₒᵤₜ in the current source unit cell with source switch 314b due the current source unit cell with source switch 314b turning on.

As described above, the current source unit cell with drain switch 214a turning on may cause a decrease in voltage at V_{bias} while the current source unit cell with source switch 314b turning on may cause an increase in voltage at V_{bias}. However, other current source unit cells with drain switch, such as the current source unit cell with drain switch 814a, may cause an increase in voltage at V_{bias}, and other current source unit cells with source switch, such as the current source unit cell with source switch 914b, may cause a decrease in voltage at V_{bias}. While the above description has focused on deriving equations for the change in voltage at V_{bias} caused by the current source unit cell with drain switch 214a and the current source unit cell with source switch 314b turning on, similar circuit analysis may be used to derive equations for the change in voltage at V_{bias} caused by other current source unit cells with different circuit topologies.

Referring back to FIG. 1 and as described above, the PLL controller 111 may control the DCO 110 to turn on the current source unit cells 114 using coarse control circuitry 113 and fine control circuitry 115. Using the coarse control circuitry 113 may include controlling the DCO 110 to turn on or off blocks of multiple current source unit cells 114 at a time. Using the fine control circuitry 115 may include controlling the DCO 110 to turn on or off single current source unit cells 114 at a time. Implementing both coarse control circuitry 113 and fine control circuitry 115 may reduce the complexity and size of circuitry needed in the control circuitry 108.

As a particular example, if a block includes four current source unit cells 114, the control circuitry 108 may use the coarse control circuitry 113 to control the DCO 110 to turn on eight blocks of current source unit cells 114 and use the fine control circuitry 115 to control the DCO 110 to turn on one additional current source unit cell 114 for a total of 33 current source unit cells 114 turned on. As another example, the control circuitry 108 may use the coarse control circuitry 113 to control the DCO 110 to turn on eight blocks of current source unit cells 114 and use the fine control circuitry 115 to control the DCO 110 to turn off one current source unit cell 114 for a total of 31 current source unit cells 114 turned on.

Consider further that, in some embodiments, the current source unit cells 114 may be ordered in a particular order, such that each current source unit cell 114 can be associated with a number 1 . . . N, where there are N total current source unit cells 114. In some embodiments, the control circuitry 108 may use the coarse control circuitry 113 and the fine control circuitry 115 such that only consecutively numbered current source unit cells 114 starting from position 1 may be turned on simultaneously. In other words, if k current source unit cells 114 are turned on, then those cells are numbered 1−k. As a particular example, if a block includes four current source unit cells 114, the control circuitry 108 may use the coarse control circuitry 113 to control the DCO 110 to turn on eight blocks of current source unit cells 114 and use the fine control circuitry 115 to control the DCO 110 to turn off one current source unit cell 114 such that current source unit cells 114 numbered 1-31 are turned on. As another example, the control circuitry 108 may use the coarse control circuitry 113 to control the DCO 110 to turn on eight blocks of current source unit cells and use the fine control circuitry 115 to control the DCO 110 to turn on one additional current source unit cell 114 such that current source unit cells 114 numbered 1-33 are turned on.

Turning on a current source unit cell with drain switch 114a having a particular topology may cause a change in voltage at V_{bias} having a certain amplitude and polarity and turning on a current source unit cell with source switch 114b having a particular topology may cause a change in voltage at V_{bias} having a certain amplitude and polarity. For example, the current source unit cell 214a may cause a decrease in voltage at V_{bias} and the current source unit cell 314b may cause an increase in voltage at V_{bias}, and the amplitude of the decrease in voltage may be less than the amplitude of the increase in voltage. However, in some embodiments, turning on a current source unit cell with drain switch 114a having a particular topology may cause an increase in voltage at V_{bias} and turning on a current source unit cell with source switch 114b having a particular topology may cause a decrease in voltage at V_{bias}. Additionally or alternatively, turning on a current source unit cell with drain switch 114a having a particular topology may cause a change in voltage at V_{bias} having a larger amplitude than the amplitude of the change in voltage caused by turning on a current source unit cell with source switch 114b having a particular topology.

The total voltage disturbance at V_{bias} caused by all the current source unit cells turning on at a particular time may depend on how many current source unit cells with drain switch 114a and how many current source unit cells with source switch 114b are turned on simultaneously, as well as the value of the decrease in voltage at V_{bias} caused by a current source unit cell with drain switch 114a turning on and the value of the increase in voltage at V_{bias} caused by a current source unit cell with source switch 114b turning on. It may be helpful if, across all the options of how many current source unit cells 114 are turned on, the total voltage disturbance at V_{bias} is, on average, as close to 0 as possible. In other words, if there are N current source unit cells 114, one may calculate the total voltage disturbance at V_{bias} when the current source unit cell 114 numbered 1 is turned on, when the current source unit cells 114 numbered 1-2 are turned on, when the current source unit cells 114 numbered 1-3 are turned on, etc. It may be helpful if the average of all the calculated total voltage disturbances is as close to 0 as possible.

The inventors have recognized that selecting a specific number of current source unit cells with drain switch 114a and the number of current source unit cells with source switch 114b in each block (i.e., a block in which all the current source unit cells are turned on or off with the coarse control circuitry 113), and selecting a specific ordering of the current source unit cells 114, may help to reduce the disturbance at V_{bias} across all options for how many current source unit cells 114 are turned on. Consider that the ratio between the decrease in voltage at V_{bias} caused by the current source unit cell with drain switch 114a turning on to the increase in voltage at V_{bias}caused by the current source unit cell with source switch 114b turning on is m:n. Then, using a size of 1+floor (n/m) current source unit cells 114 for each, (or approximately each) block and a block composition of floor (n/m) current source unit cells with drain switch 114a and 1 current source unit cell with source switch 114b may further help to reduce the disturbance at the V_{bias} node.

Table 1 illustrates two example block sizes and compositions for current source unit cells 114 where the decrease in voltage at V_{bias} caused by the current source unit cell with drain switch 114a turning on (m) is −4.87 mV and the increase in voltage at V_{bias} caused by the current source unit cell with source switch 114b turning on is 15.21 mV (n), such that the ratio m:n is 1:3.13. Case 2 of Table 1 illustrates an ordering of current source unit cells 114 using the above-described block size of 1+floor (n/m)=4 current source unit cells 114 and a block composition of floor (n/m)=3 current source unit cells with drain switch 114a and 1 current source unit cell with source switch 114b. Case 1 illustrates a different ordering that does not use the above-described block size or composition, but instead has a block size of 32 and a block composition of 24 current source unit cells with drain switch 114a and 8 current source unit cell with source switch 114b. Each row lists, in one column, whether the current source unit 114 at a particular position n in the order is a current source unit cell with drain switch 114a (“D”) or current source unit cell with source switch 114b (“S”). As can be seen, the ordering in Case 1 and Case 2 both include, in each block, all the current source unit cells with drain switch 114a (“D”) positioned first followed by all the current source unit cells with source switch 114b (“S”). Each row lists in the other column the sum of the voltage disturbances (in mV) caused at V_{bias} by the current source units 114 numbered 1−k when turned on. In other words, if the current source unit cells 114 numbered 1−k include d current source unit cells with drain switch 114a each contributing −4.87 mV to V_{bias} and s current source unit cells with source switch 114b each contributing 15.21 mV to V_{bias}, then the total at each row may be 15.21 s-4.87 d. Generally, if the current source unit cells 114 numbered 1−k include d current source unit cells with drain switch 114a each contributing m to V_{bias} and s current source unit cells with source switch 114b each contributing n to V_{bias} then the total at each row may be ns−md.

When using a block composition of floor (n/m) current source unit cells with drain switch 114a and 1 current source unit cell with source switch 114b, there may be an error of (n/m−floor (n/m)). In the example above, the ratio m:n is 1:3.13. The block composition of 3 current source unit cells with drain switch 114a and 1 current source unit cells with source switch 114b may not account for the extra 0.13. In some embodiments, to compensate for this error, a current source unit cell with drain switch 114a may be added approximately every round ((1+floor (n/m))/(n/m−floor (n/m)) positions in the order. For example, in the above example, a current source unit cell with drain switch 114a may be added approximately every round (4/0.13)=30.77 positions in the order, or in practice, alternating every 31^{st} and 30^{th} positions in the order. Case 3 of Table 1 illustrates an ordering of current source unit cells 114 that is the same as Case 2, except that Case 3 includes an extra current source unit cell with drain switch 114a (“D”) added every 31^{st} or 30^{th} positions (alternating). The extra current source unit cell with drain switch 114a is illustrated with a bold “D”.

The total voltage disturbance may be further improved if the current source unit cell with source switch 114b (“S”) in each block is approximately in the middle of the block. For example, Case 4 illustrates an ordering that is the same as Case 3 but offset by 2 positions (in other words, starting from position 3 of Case 3) such that, in each block of size 4, the current source unit cell with source switch 114b (“S”) is at the second position rather than the last position (as it is in Case 3).

In general, Case 1 may be an ordering that has been optimized according to any of the criteria described above. Case 2 may be an ordering using a block size of 1+floor (n/m) and a block composition of floor (n/m)=current source unit cells with drain switch 114a and 1 current source unit cell with source switch 114b. Case 3 may be the same as Case 2 but with a current source unit cell with drain switch 114a added approximately every round ((1+floor (n/m))/(n/m−floor (n/m)) positions in the order. Case 4 may be the same as Case 3 but offset such that the current source unit cell with source switch 114b in each block is positioned approximately in the middle of each block.

In some embodiments, an ultrasound device may be hard coded based on the values of table 1. In other embodiments, table 1 may be stored as a look-up table in a memory of an ultrasound device (or a memory outside the ultrasound device). In some embodiments, determining how many current sources of type “D” (e.g., with drain switches) and how many current sources of type “S” (e.g., with source switches) should be turned on may involve identifying a plurality of entries (e.g., a pair of entries) of the look-up table, that, collectively, yield a voltage disturbance below a certain threshold.

TABLE 1
Four different orderings of current source unit cells 114 and
the total voltage disturbance caused to V_{bias} as a function
of how many current source unit cells 114 are turned on.

| # of Cells | | | | | | | | |
|---|---|---|---|---|---|---|---|---|
| Turned On | Case 1 | | Case 2 | | Case 3 | | Case 4 | |
| 1 | D | −4.87 | D | −4.87 | D | −4.87 | D | −4.87 |
| 2 | D | −9.74 | D | −9.74 | D | −9.74 | S | 10.34 |
| 3 | D | −14.61 | D | −14.61 | D | −14.61 | D | 5.47 |
| 4 | D | −19.48 | S | 0.6 | S | 0.6 | D | 0.6 |
| 5 | D | −24.35 | D | −4.27 | D | −4.27 | D | −4.27 |
| 6 | D | −29.22 | D | −9.14 | D | −9.14 | S | 10.94 |
| 7 | D | −34.09 | D | −14.01 | D | −14.01 | D | 6.07 |
| 8 | D | −38.96 | S | 1.2 | S | 1.2 | D | 1.2 |
| 9 | D | −43.83 | D | −3.67 | D | −3.67 | D | −3.67 |
| 10 | D | −48.7 | D | −8.54 | D | −8.54 | S | 11.54 |
| 11 | D | −53.57 | D | −13.41 | D | −13.41 | D | 6.67 |
| 12 | D | −58.44 | S | 1.8 | S | 1.8 | D | 1.8 |
| 13 | D | −63.31 | D | −3.07 | D | −3.07 | D | −3.07 |
| 14 | D | −68.18 | D | −7.94 | D | −7.94 | S | 12.14 |
| 15 | D | −73.05 | D | −12.81 | D | −12.81 | D | 7.27 |
| 16 | D | −77.92 | S | 2.4 | S | 2.4 | D | 2.4 |
| 17 | D | −82.79 | D | −2.47 | D | −2.47 | D | −2.47 |
| 18 | D | −87.66 | D | −7.34 | D | −7.34 | S | 12.74 |
| 19 | D | −92.53 | D | −12.21 | D | −12.21 | D | 7.87 |
| 20 | D | −97.4 | S | 3 | S | 3 | D | 3 |
| 21 | D | −102.27 | D | −1.87 | D | −1.87 | D | −1.87 |
| 22 | D | −107.14 | D | −6.74 | D | −6.74 | S | 13.34 |
| 23 | D | −112.01 | D | −11.61 | D | −11.61 | D | 8.47 |
| 24 | D | −116.88 | S | 3.6 | S | 3.6 | D | 3.6 |
| 25 | S | −101.67 | D | −1.27 | D | −1.27 | D | −1.27 |
| 26 | S | −86.46 | D | −6.14 | D | −6.14 | S | 13.94 |
| 27 | S | −71.25 | D | −11.01 | D | −11.01 | D | 9.07 |
| 28 | S | −56.04 | S | 4.2 | S | 4.2 | D | 4.2 |
| 29 | S | −40.83 | D | −0.67 | D | −0.67 | D | −0.67 |
| 30 | S | −25.62 | D | −5.54 | D | −5.54 | D | −5.54 |
| 31 | S | −10.41 | D | −10.41 | D | −10.41 | S | 9.67 |
| 32 | S | 4.8 | S | 4.8 | D | −15.28 | D | 4.8 |
| 33 | D | −0.07 | D | −0.07 | S | −0.07 | D | −0.07 |
| 34 | D | −4.94 | D | −4.94 | D | −4.94 | D | −4.94 |
| 35 | D | −9.81 | D | −9.81 | D | −9.81 | S | 10.27 |
| 36 | D | −14.68 | S | 5.4 | D | −14.68 | D | 5.4 |
| 37 | D | −19.55 | D | 0.53 | S | 0.53 | D | 0.53 |
| 38 | D | −24.42 | D | −4.34 | D | −4.34 | D | −4.34 |
| 39 | D | −29.29 | D | −9.21 | D | −9.21 | S | 10.87 |
| 40 | D | −34.16 | S | 6 | D | −14.08 | D | 6 |
| 41 | D | −39.03 | D | 1.13 | S | 1.13 | D | 1.13 |
| 42 | D | −43.9 | D | −3.74 | D | −3.74 | D | −3.74 |
| 43 | D | −48.77 | D | −8.61 | D | −8.61 | S | 11.47 |
| 44 | D | −53.64 | S | 6.6 | D | −13.48 | D | 6.6 |
| 45 | D | −58.51 | D | 1.73 | S | 1.73 | D | 1.73 |
| 46 | D | −63.38 | D | −3.14 | D | −3.14 | D | −3.14 |
| 47 | D | −68.25 | D | −8.01 | D | −8.01 | S | 12.07 |
| 48 | D | −73.12 | S | 7.2 | D | −12.88 | D | 7.2 |
| 49 | D | −77.99 | D | 2.33 | S | 2.33 | D | 2.33 |
| 50 | D | −82.86 | D | −2.54 | D | −2.54 | D | −2.54 |
| 51 | D | −87.73 | D | −7.41 | D | −7.41 | S | 12.67 |
| 52 | D | −92.6 | S | 7.8 | D | −12.28 | D | 7.8 |
| 53 | D | −97.47 | D | 2.93 | S | 2.93 | D | 2.93 |
| 54 | D | −102.34 | D | −1.94 | D | −1.94 | D | −1.94 |
| 55 | D | −107.21 | D | −6.81 | D | −6.81 | S | 13.27 |
| 56 | D | −112.08 | S | 8.4 | D | −11.68 | D | 8.4 |
| 57 | S | −96.87 | D | 3.53 | S | 3.53 | D | 3.53 |
| 58 | S | −81.66 | D | −1.34 | D | −1.34 | D | −1.34 |
| 59 | S | −66.45 | D | −6.21 | D | −6.21 | D | −6.21 |
| 60 | S | −51.24 | S | 9 | D | −11.08 | S | 9 |
| 61 | S | −36.03 | D | 4.13 | D | −15.95 | D | 4.13 |
| 62 | S | −20.82 | D | −0.74 | S | −0.74 | D | −0.74 |
| 63 | S | −5.61 | D | −5.61 | D | −5.61 | D | −5.61 |
| 64 | S | 9.6 | S | 9.6 | D | −10.48 | S | 9.6 |
| 65 | D | 4.73 | D | 4.73 | D | −15.35 | D | 4.73 |
| 66 | D | −0.14 | D | −0.14 | S | −0.14 | D | −0.14 |
| 67 | D | −5.01 | D | −5.01 | D | −5.01 | D | −5.01 |
| 68 | D | −9.88 | S | 10.2 | D | −9.88 | S | 10.2 |
| 69 | D | −14.75 | D | 5.33 | D | −14.75 | D | 5.33 |
| 70 | D | −19.62 | D | 0.46 | S | 0.46 | D | 0.46 |
| 71 | D | −24.49 | D | −4.41 | D | −4.41 | D | −4.41 |
| 72 | D | −29.36 | S | 10.8 | D | −9.28 | S | 10.8 |
| 73 | D | −34.23 | D | 5.93 | D | −14.15 | D | 5.93 |
| 74 | D | −39.1 | D | 1.06 | S | 1.06 | D | 1.06 |
| 75 | D | −43.97 | D | −3.81 | D | −3.81 | D | −3.81 |
| 76 | D | −48.84 | S | 11.4 | D | −8.68 | S | 11.4 |
| 77 | D | −53.71 | D | 6.53 | D | −13.55 | D | 6.53 |
| 78 | D | −58.58 | D | 1.66 | S | 1.66 | D | 1.66 |
| 79 | D | −63.45 | D | −3.21 | D | −3.21 | D | −3.21 |
| 80 | D | −68.32 | S | 12 | D | −8.08 | S | 12 |
| 81 | D | −73.19 | D | 7.13 | D | −12.95 | D | 7.13 |
| 82 | D | −78.06 | D | 2.26 | S | 2.26 | D | 2.26 |
| 83 | D | −82.93 | D | −2.61 | D | −2.61 | D | −2.61 |
| 84 | D | −87.8 | S | 12.6 | D | −7.48 | S | 12.6 |
| 85 | D | −92.67 | D | 7.73 | D | −12.35 | D | 7.73 |
| 86 | D | −97.54 | D | 2.86 | S | 2.86 | D | 2.86 |
| 87 | D | −102.41 | D | −2.01 | D | −2.01 | D | −2.01 |
| 88 | D | −107.28 | S | 13.2 | D | −6.88 | S | 13.2 |
| 89 | S | −92.07 | D | 8.33 | D | −11.75 | D | 8.33 |
| 90 | S | −76.86 | D | 3.46 | S | 3.46 | D | 3.46 |
| 91 | S | −61.65 | D | −1.41 | D | −1.41 | D | −1.41 |
| 92 | S | −46.44 | S | 13.8 | D | −6.28 | D | −6.28 |
| 93 | S | −31.23 | D | 8.93 | D | −11.15 | S | 8.93 |
| 94 | S | −16.02 | D | 4.06 | D | −16.02 | D | 4.06 |
| 95 | S | −0.81 | D | −0.81 | S | −0.81 | D | −0.81 |
| 96 | S | 14.4 | S | 14.4 | D | −5.68 | D | −5.68 |
| 97 | D | 9.53 | D | 9.53 | D | −10.55 | S | 9.53 |
| 98 | D | 4.66 | D | 4.66 | D | −15.42 | D | 4.66 |
| 99 | D | −0.21 | D | −0.21 | S | −0.21 | D | −0.21 |
| 100 | D | −5.08 | S | 15 | D | −5.08 | D | −5.08 |
| 101 | D | −9.95 | D | 10.13 | D | −9.95 | S | 10.13 |
| 102 | D | −14.82 | D | 5.26 | D | −14.82 | D | 5.26 |
| 103 | D | −19.69 | D | 0.39 | S | 0.39 | D | 0.39 |
| 104 | D | −24.56 | S | 15.6 | D | −4.48 | D | −4.48 |
| 105 | D | −29.43 | D | 10.73 | D | −9.35 | S | 10.73 |
| 106 | D | −34.3 | D | 5.86 | D | −14.22 | D | 5.86 |
| 107 | D | −39.17 | D | 0.99 | S | 0.99 | D | 0.99 |
| 108 | D | −44.04 | S | 16.2 | D | −3.88 | D | −3.88 |
| 109 | D | −48.91 | D | 11.33 | D | −8.75 | S | 11.33 |
| 110 | D | −53.78 | D | 6.46 | D | −13.62 | D | 6.46 |
| 111 | D | −58.65 | D | 1.59 | S | 1.59 | D | 1.59 |
| 112 | D | −63.52 | S | 16.8 | D | −3.28 | D | −3.28 |
| 113 | D | −68.39 | D | 11.93 | D | −8.15 | S | 11.93 |
| 114 | D | −73.26 | D | 7.06 | D | −13.02 | D | 7.06 |
| 115 | D | −78.13 | D | 2.19 | S | 2.19 | D | 2.19 |
| 116 | D | −83 | S | 17.4 | D | −2.68 | D | −2.68 |
| 117 | D | −87.87 | D | 12.53 | D | −7.55 | S | 12.53 |
| 118 | D | −92.74 | D | 7.66 | D | −12.42 | D | 7.66 |
| 119 | D | −97.61 | D | 2.79 | S | 2.79 | D | 2.79 |
| 120 | D | −102.48 | S | 18 | D | −2.08 | D | −2.08 |
| 121 | S | −87.27 | D | 13.13 | D | −6.95 | D | −6.95 |
| 122 | S | −72.06 | D | 8.26 | D | −11.82 | S | 8.26 |
| 123 | S | −56.85 | D | 3.39 | D | −16.69 | D | 3.39 |
| 124 | S | −41.64 | S | 18.6 | S | −1.48 | D | −1.48 |
| 125 | S | −26.43 | D | 13.73 | D | −6.35 | D | −6.35 |
| 126 | S | −11.22 | D | 8.86 | D | −11.22 | S | 8.86 |
| 127 | S | 3.99 | D | 3.99 | D | −16.09 | D | 3.99 |
| 128 | S | 19.2 | S | 19.2 | S | −0.88 | D | −0.88 |
| 129 | D | 14.33 | D | 14.33 | D | −5.75 | D | −5.75 |
| 130 | D | 9.46 | D | 9.46 | D | −10.62 | S | 9.46 |
| 131 | D | 4.59 | D | 4.59 | D | −15.49 | D | 4.59 |
| 132 | D | −0.28 | S | 19.8 | S | −0.28 | D | −0.28 |
| 133 | D | −5.15 | D | 14.93 | D | −5.15 | D | −5.15 |
| 134 | D | −10.02 | D | 10.06 | D | −10.02 | S | 10.06 |
| 135 | D | −14.89 | D | 5.19 | D | −14.89 | D | 5.19 |
| 136 | D | −19.76 | S | 20.4 | S | 0.32 | D | 0.32 |
| 137 | D | −24.63 | D | 15.53 | D | −4.55 | D | −4.55 |
| 138 | D | −29.5 | D | 10.66 | D | −9.42 | S | 10.66 |
| 139 | D | −34.37 | D | 5.79 | D | −14.29 | D | 5.79 |
| 140 | D | −39.24 | S | 21 | S | 0.92 | D | 0.92 |
| 141 | D | −44.11 | D | 16.13 | D | −3.95 | D | −3.95 |
| 142 | D | −48.98 | D | 11.26 | D | −8.82 | S | 11.26 |
| 143 | D | −53.85 | D | 6.39 | D | −13.69 | D | 6.39 |
| 144 | D | −58.72 | S | 21.6 | S | 1.52 | D | 1.52 |
| 145 | D | −63.59 | D | 16.73 | D | −3.35 | D | −3.35 |
| 146 | D | −68.46 | D | 11.86 | D | −8.22 | S | 11.86 |
| 147 | D | −73.33 | D | 6.99 | D | −13.09 | D | 6.99 |
| 148 | D | −78.2 | S | 22.2 | S | 2.12 | D | 2.12 |
| 149 | D | −83.07 | D | 17.33 | D | −2.75 | D | −2.75 |
| 150 | D | −87.94 | D | 12.46 | D | −7.62 | S | 12.46 |
| 151 | D | −92.81 | D | 7.59 | D | −12.49 | D | 7.59 |
| 152 | D | −97.68 | S | 22.8 | S | 2.72 | D | 2.72 |
| 153 | S | −82.47 | D | 17.93 | D | −2.15 | D | −2.15 |
| 154 | S | −67.26 | D | 13.06 | D | −7.02 | D | −7.02 |
| 155 | S | −52.05 | D | 8.19 | D | −11.89 | S | 8.19 |
| 156 | S | −36.84 | S | 23.4 | D | −16.76 | D | 3.32 |
| 157 | S | −21.63 | D | 18.53 | S | −1.55 | D | −1.55 |
| 158 | S | −6.42 | D | 13.66 | D | −6.42 | D | −6.42 |
| 159 | S | 8.79 | D | 8.79 | D | −11.29 | S | 8.79 |
| 160 | S | 24 | S | 24 | D | −16.16 | D | 3.92 |
| 161 | D | 19.13 | D | 19.13 | S | −0.95 | D | −0.95 |
| 162 | D | 14.26 | D | 14.26 | D | −5.82 | D | −5.82 |
| 163 | D | 9.39 | D | 9.39 | D | −10.69 | S | 9.39 |
| 164 | D | 4.52 | S | 24.6 | D | −15.56 | D | 4.52 |
| 165 | D | −0.35 | D | 19.73 | S | −0.35 | D | −0.35 |
| 166 | D | −5.22 | D | 14.86 | D | −5.22 | D | −5.22 |
| 167 | D | −10.09 | D | 9.99 | D | −10.09 | S | 9.99 |
| 168 | D | −14.96 | S | 25.2 | D | −14.96 | D | 5.12 |
| 169 | D | −19.83 | D | 20.33 | S | 0.25 | D | 0.25 |
| 170 | D | −24.7 | D | 15.46 | D | −4.62 | D | −4.62 |
| 171 | D | −29.57 | D | 10.59 | D | −9.49 | S | 10.59 |
| 172 | D | −34.44 | S | 25.8 | D | −14.36 | D | 5.72 |
| 173 | D | −39.31 | D | 20.93 | S | 0.85 | D | 0.85 |
| 174 | D | −44.18 | D | 16.06 | D | −4.02 | D | −4.02 |
| 175 | D | −49.05 | D | 11.19 | D | −8.89 | S | 11.19 |
| 176 | D | −53.92 | S | 26.4 | D | −13.76 | D | 6.32 |
| 177 | D | −58.79 | D | 21.53 | S | 1.45 | D | 1.45 |
| 178 | D | −63.66 | D | 16.66 | D | −3.42 | D | −3.42 |
| 179 | D | −68.53 | D | 11.79 | D | −8.29 | S | 11.79 |
| 180 | D | −73.4 | S | 27 | D | −13.16 | D | 6.92 |
| 181 | D | −78.27 | D | 22.13 | S | 2.05 | D | 2.05 |
| 182 | D | −83.14 | D | 17.26 | D | −2.82 | D | −2.82 |
| 183 | D | −88.01 | D | 12.39 | D | −7.69 | D | −7.69 |
| 184 | D | −92.88 | S | 27.6 | D | −12.56 | S | 7.52 |
| 185 | S | −77.67 | D | 22.73 | D | −17.43 | D | 2.65 |
| 186 | S | −62.46 | D | 17.86 | S | −2.22 | D | −2.22 |
| 187 | S | −47.25 | D | 12.99 | D | −7.09 | D | −7.09 |
| 188 | S | −32.04 | S | 28.2 | D | −11.96 | S | 8.12 |
| 189 | S | −16.83 | D | 23.33 | D | −16.83 | D | 3.25 |
| 190 | S | −1.62 | D | 18.46 | S | −1.62 | D | −1.62 |
| 191 | S | 13.59 | D | 13.59 | D | −6.49 | D | −6.49 |
| 192 | S | 28.8 | S | 28.8 | D | −11.36 | S | 8.72 |
| 193 | D | 23.93 | D | 23.93 | D | −16.23 | D | 3.85 |
| 194 | D | 19.06 | D | 19.06 | S | −1.02 | D | −1.02 |
| 195 | D | 14.19 | D | 14.19 | D | −5.89 | D | −5.89 |
| 196 | D | 9.32 | S | 29.4 | D | −10.76 | S | 9.32 |
| 197 | D | 4.45 | D | 24.53 | D | −15.63 | D | 4.45 |
| 198 | D | −0.42 | D | 19.66 | S | −0.42 | D | −0.42 |
| 199 | D | −5.29 | D | 14.79 | D | −5.29 | D | −5.29 |
| 200 | D | −10.16 | S | 30 | D | −10.16 | S | 9.92 |
| 201 | D | −15.03 | D | 25.13 | D | −15.03 | D | 5.05 |
| 202 | D | −19.9 | D | 20.26 | S | 0.18 | D | 0.18 |
| 203 | D | −24.77 | D | 15.39 | D | −4.69 | D | −4.69 |
| 204 | D | −29.64 | S | 30.6 | D | −9.56 | S | 10.52 |
| 205 | D | −34.51 | D | 25.73 | D | −14.43 | D | 5.65 |
| 206 | D | −39.38 | D | 20.86 | S | 0.78 | D | 0.78 |
| 207 | D | −44.25 | D | 15.99 | D | −4.09 | D | −4.09 |
| 208 | D | −49.12 | S | 31.2 | D | −8.96 | S | 11.12 |
| 209 | D | −53.99 | D | 26.33 | D | −13.83 | D | 6.25 |
| 210 | D | −58.86 | D | 21.46 | S | 1.38 | D | 1.38 |
| 211 | D | −63.73 | D | 16.59 | D | −3.49 | D | −3.49 |
| 212 | D | −68.6 | S | 31.8 | D | −8.36 | D | −8.36 |
| 213 | D | −73.47 | D | 26.93 | D | −13.23 | S | 6.85 |
| 214 | D | −78.34 | D | 22.06 | D | −18.1 | D | 1.98 |
| 215 | D | −83.21 | D | 17.19 | S | −2.89 | D | −2.89 |
| 216 | D | −88.08 | S | 32.4 | D | −7.76 | D | −7.76 |
| 217 | S | −72.87 | D | 27.53 | D | −12.63 | S | 7.45 |
| 218 | S | −57.66 | D | 22.66 | D | −17.5 | D | 2.58 |
| 219 | S | −42.45 | D | 17.79 | S | −2.29 | D | −2.29 |
| 220 | S | −27.24 | S | 33 | D | −7.16 | D | −7.16 |
| 221 | S | −12.03 | D | 28.13 | D | −12.03 | S | 8.05 |
| 222 | S | 3.18 | D | 23.26 | D | −16.9 | D | 3.18 |
| 223 | S | 18.39 | D | 18.39 | S | −1.69 | D | −1.69 |
| 224 | S | 33.6 | S | 33.6 | D | −6.56 | D | −6.56 |
| 225 | D | 28.73 | D | 28.73 | D | −11.43 | S | 8.65 |
| 226 | D | 23.86 | D | 23.86 | D | −16.3 | D | 3.78 |
| 227 | D | 18.99 | D | 18.99 | S | −1.09 | D | −1.09 |
| 228 | D | 14.12 | S | 34.2 | D | −5.96 | D | −5.96 |
| 229 | D | 9.25 | D | 29.33 | D | −10.83 | S | 9.25 |
| 230 | D | 4.38 | D | 24.46 | D | −15.7 | D | 4.38 |
| 231 | D | −0.49 | D | 19.59 | S | −0.49 | D | −0.49 |
| 232 | D | −5.36 | S | 34.8 | D | −5.36 | D | −5.36 |
| 233 | D | −10.23 | D | 29.93 | D | −10.23 | S | 9.85 |
| 234 | D | −15.1 | D | 25.06 | D | −15.1 | D | 4.98 |
| 235 | D | −19.97 | D | 20.19 | S | 0.11 | D | 0.11 |
| 236 | D | −24.84 | S | 35.4 | D | −4.76 | D | −4.76 |
| 237 | D | −29.71 | D | 30.53 | D | −9.63 | S | 10.45 |
| 238 | D | −34.58 | D | 25.66 | D | −14.5 | D | 5.58 |
| 239 | D | −39.45 | D | 20.79 | S | 0.71 | D | 0.71 |
| 240 | D | −44.32 | S | 36 | D | −4.16 | D | −4.16 |
| 241 | D | −49.19 | D | 31.13 | D | −9.03 | S | 11.05 |
| 242 | D | −54.06 | D | 26.26 | D | −13.9 | D | 6.18 |
| 243 | D | −58.93 | D | 21.39 | S | 1.31 | D | 1.31 |
| 244 | D | −63.8 | S | 36.6 | D | −3.56 | D | −3.56 |
| 245 | D | −68.67 | D | 31.73 | D | −8.43 | D | −8.43 |
| 246 | D | −73.54 | D | 26.86 | D | −13.3 | S | 6.78 |
| 247 | D | −78.41 | D | 21.99 | D | −18.17 | D | 1.91 |
| 248 | D | −83.28 | S | 37.2 | S | −2.96 | D | −2.96 |
| 249 | S | −68.07 | D | 32.33 | D | −7.83 | D | −7.83 |
| 250 | S | −52.86 | D | 27.46 | D | −12.7 | S | 7.38 |
| 251 | S | −37.65 | D | 22.59 | D | −17.57 | D | 2.51 |
| 252 | S | −22.44 | S | 37.8 | S | −2.36 | D | −2.36 |
| 253 | S | −7.23 | D | 32.93 | D | −7.23 | D | −7.23 |
| 254 | S | 7.98 | D | 28.06 | D | −12.1 | S | 7.98 |
| 255 | S | 23.19 | D | 23.19 | D | −16.97 | D | 3.11 |
| 256 | S | 38.4 | S | 38.4 | S | −1.76 | D | −1.76 |
| 257 | D | 33.53 | D | 33.53 | D | −6.63 | D | −6.63 |
| 258 | D | 28.66 | D | 28.66 | D | −11.5 | S | 8.58 |
| 259 | D | 23.79 | D | 23.79 | D | −16.37 | D | 3.71 |
| 260 | D | 18.92 | S | 39 | S | −1.16 | D | −1.16 |
| 261 | D | 14.05 | D | 34.13 | D | −6.03 | D | −6.03 |
| 262 | D | 9.18 | D | 29.26 | D | −10.9 | S | 9.18 |
| 263 | D | 4.31 | D | 24.39 | D | −15.77 | D | 4.31 |
| 264 | D | −0.56 | S | 39.6 | S | −0.56 | D | −0.56 |
| 265 | D | −5.43 | D | 34.73 | D | −5.43 | D | −5.43 |
| 266 | D | −10.3 | D | 29.86 | D | −10.3 | S | 9.78 |
| 267 | D | −15.17 | D | 24.99 | D | −15.17 | D | 4.91 |
| 268 | D | −20.04 | S | 40.2 | S | 0.04 | D | 0.04 |
| 269 | D | −24.91 | D | 35.33 | D | −4.83 | D | −4.83 |
| 270 | D | −29.78 | D | 30.46 | D | −9.7 | S | 10.38 |
| 271 | D | −34.65 | D | 25.59 | D | −14.57 | D | 5.51 |
| 272 | D | −39.52 | S | 40.8 | S | 0.64 | D | 0.64 |
| 273 | D | −44.39 | D | 35.93 | D | −4.23 | D | −4.23 |
| 274 | D | −49.26 | D | 31.06 | D | −9.1 | D | −9.1 |
| 275 | D | −54.13 | D | 26.19 | D | −13.97 | S | 6.11 |
| 276 | D | −59 | S | 41.4 | D | −18.84 | D | 1.24 |
| 277 | D | −63.87 | D | 36.53 | S | −3.63 | D | −3.63 |
| 278 | D | −68.74 | D | 31.66 | D | −8.5 | D | −8.5 |
| 279 | D | −73.61 | D | 26.79 | D | −13.37 | S | 6.71 |
| 280 | D | −78.48 | S | 42 | D | −18.24 | D | 1.84 |
| 281 | S | −63.27 | D | 37.13 | S | −3.03 | D | −3.03 |
| 282 | S | −48.06 | D | 32.26 | D | −7.9 | D | −7.9 |
| 283 | S | −32.85 | D | 27.39 | D | −12.77 | S | 7.31 |
| 284 | S | −17.64 | S | 42.6 | D | −17.64 | D | 2.44 |
| 285 | S | −2.43 | D | 37.73 | S | −2.43 | D | −2.43 |
| 286 | S | 12.78 | D | 32.86 | D | −7.3 | D | −7.3 |
| 287 | S | 27.99 | D | 27.99 | D | −12.17 | S | 7.91 |
| 288 | S | 43.2 | S | 43.2 | D | −17.04 | D | 3.04 |
| 289 | D | 38.33 | D | 38.33 | S | −1.83 | D | −1.83 |
| 290 | D | 33.46 | D | 33.46 | D | −6.7 | D | −6.7 |
| 291 | D | 28.59 | D | 28.59 | D | −11.57 | S | 8.51 |
| 292 | D | 23.72 | S | 43.8 | D | −16.44 | D | 3.64 |
| 293 | D | 18.85 | D | 38.93 | S | −1.23 | D | −1.23 |
| 294 | D | 13.98 | D | 34.06 | D | −6.1 | D | −6.1 |
| 295 | D | 9.11 | D | 29.19 | D | −10.97 | S | 9.11 |
| 296 | D | 4.24 | S | 44.4 | D | −15.84 | D | 4.24 |
| 297 | D | −0.63 | D | 39.53 | S | −0.63 | D | −0.63 |
| 298 | D | −5.5 | D | 34.66 | D | −5.5 | D | −5.5 |
| 299 | D | −10.37 | D | 29.79 | D | −10.37 | S | 9.71 |
| 300 | D | −15.24 | S | 45 | D | −15.24 | D | 4.84 |
| 301 | D | −20.11 | D | 40.13 | S | −0.03 | D | −0.03 |
| 302 | D | −24.98 | D | 35.26 | D | −4.9 | D | −4.9 |
| 303 | D | −29.85 | D | 30.39 | D | −9.77 | D | −9.77 |
| 304 | D | −34.72 | S | 45.6 | D | −14.64 | S | 5.44 |
| 305 | D | −39.59 | D | 40.73 | D | −19.51 | D | 0.57 |
| 306 | D | −44.46 | D | 35.86 | S | −4.3 | D | −4.3 |
| 307 | D | −49.33 | D | 30.99 | D | −9.17 | D | −9.17 |
| 308 | D | −54.2 | S | 46.2 | D | −14.04 | S | 6.04 |
| 309 | D | −59.07 | D | 41.33 | D | −18.91 | D | 1.17 |
| 310 | D | −63.94 | D | 36.46 | S | −3.7 | D | −3.7 |
| 311 | D | −68.81 | D | 31.59 | D | −8.57 | D | −8.57 |
| 312 | D | −73.68 | S | 46.8 | D | −13.44 | S | 6.64 |
| 313 | S | −58.47 | D | 41.93 | D | −18.31 | D | 1.77 |
| 314 | S | −43.26 | D | 37.06 | S | −3.1 | D | −3.1 |
| 315 | S | −28.05 | D | 32.19 | D | −7.97 | D | −7.97 |
| 316 | S | −12.84 | S | 47.4 | D | −12.84 | S | 7.24 |
| 317 | S | 2.37 | D | 42.53 | D | −17.71 | D | 2.37 |
| 318 | S | 17.58 | D | 37.66 | S | −2.5 | D | −2.5 |
| 319 | S | 32.79 | D | 32.79 | D | −7.37 | D | −7.37 |
| 320 | S | 48 | S | 48 | D | −12.24 | S | 7.84 |
| 321 | D | 43.13 | D | 43.13 | D | −17.11 | D | 2.97 |
| 322 | D | 38.26 | D | 38.26 | S | −1.9 | D | −1.9 |
| 323 | D | 33.39 | D | 33.39 | D | −6.77 | D | −6.77 |
| 324 | D | 28.52 | S | 48.6 | D | −11.64 | S | 8.44 |
| 325 | D | 23.65 | D | 43.73 | D | −16.51 | D | 3.57 |
| 326 | D | 18.78 | D | 38.86 | S | −1.3 | D | −1.3 |
| 327 | D | 13.91 | D | 33.99 | D | −6.17 | D | −6.17 |
| 328 | D | 9.04 | S | 49.2 | D | −11.04 | S | 9.04 |
| 329 | D | 4.17 | D | 44.33 | D | −15.91 | D | 4.17 |
| 330 | D | −0.7 | D | 39.46 | S | −0.7 | D | −0.7 |
| 331 | D | −5.57 | D | 34.59 | D | −5.57 | D | −5.57 |
| 332 | D | −10.44 | S | 49.8 | D | −10.44 | S | 9.64 |
| 333 | D | −15.31 | D | 44.93 | D | −15.31 | D | 4.77 |
| 334 | D | −20.18 | D | 40.06 | S | −0.1 | D | −0.1 |
| 335 | D | −25.05 | D | 35.19 | D | −4.97 | D | −4.97 |
| 336 | D | −29.92 | S | 50.4 | D | −9.84 | D | −9.84 |
| 337 | D | −34.79 | D | 45.53 | D | −14.71 | S | 5.37 |
| 338 | D | −39.66 | D | 40.66 | D | −19.58 | D | 0.5 |
| 339 | D | −44.53 | D | 35.79 | S | −4.37 | D | −4.37 |
| 340 | D | −49.4 | S | 51 | D | −9.24 | D | −9.24 |
| 341 | D | −54.27 | D | 46.13 | D | −14.11 | S | 5.97 |
| 342 | D | −59.14 | D | 41.26 | D | −18.98 | D | 1.1 |
| 343 | D | −64.01 | D | 36.39 | S | −3.77 | D | −3.77 |
| 344 | D | −68.88 | S | 51.6 | D | −8.64 | D | −8.64 |
| 345 | S | −53.67 | D | 46.73 | D | −13.51 | S | 6.57 |
| 346 | S | −38.46 | D | 41.86 | D | −18.38 | D | 1.7 |
| 347 | S | −23.25 | D | 36.99 | S | −3.17 | D | −3.17 |
| 348 | S | −8.04 | S | 52.2 | D | −8.04 | D | −8.04 |
| 349 | S | 7.17 | D | 47.33 | D | −12.91 | S | 7.17 |
| 350 | S | 22.38 | D | 42.46 | D | −17.78 | D | 2.3 |
| 351 | S | 37.59 | D | 37.59 | S | −2.57 | D | −2.57 |
| 352 | S | 52.8 | S | 52.8 | D | −7.44 | D | −7.44 |
| 353 | D | 47.93 | D | 47.93 | D | −12.31 | S | 7.77 |
| 354 | D | 43.06 | D | 43.06 | D | −17.18 | D | 2.9 |
| 355 | D | 38.19 | D | 38.19 | S | −1.97 | D | −1.97 |
| 356 | D | 33.32 | S | 53.4 | D | −6.84 | D | −6.84 |
| 357 | D | 28.45 | D | 48.53 | D | −11.71 | S | 8.37 |
| 358 | D | 23.58 | D | 43.66 | D | −16.58 | D | 3.5 |
| 359 | D | 18.71 | D | 38.79 | S | −1.37 | D | −1.37 |
| 360 | D | 13.84 | S | 54 | D | −6.24 | D | −6.24 |
| 361 | D | 8.97 | D | 49.13 | D | −11.11 | S | 8.97 |
| 362 | D | 4.1 | D | 44.26 | D | −15.98 | D | 4.1 |
| 363 | D | −0.77 | D | 39.39 | S | −0.77 | D | −0.77 |
| 364 | D | −5.64 | S | 54.6 | D | −5.64 | D | −5.64 |
| 365 | D | −10.51 | D | 49.73 | D | −10.51 | D | −10.51 |
| 366 | D | −15.38 | D | 44.86 | D | −15.38 | S | 4.7 |
| 367 | D | −20.25 | D | 39.99 | D | −20.25 | D | −0.17 |
| 368 | D | −25.12 | S | 55.2 | S | −5.04 | D | −5.04 |
| 369 | D | −29.99 | D | 50.33 | D | −9.91 | D | −9.91 |
| 370 | D | −34.86 | D | 45.46 | D | −14.78 | S | 5.3 |
| 371 | D | −39.73 | D | 40.59 | D | −19.65 | D | 0.43 |
| 372 | D | −44.6 | S | 55.8 | S | −4.44 | D | −4.44 |
| 373 | D | −49.47 | D | 50.93 | D | −9.31 | D | −9.31 |
| 374 | D | −54.34 | D | 46.06 | D | −14.18 | S | 5.9 |
| 375 | D | −59.21 | D | 41.19 | D | −19.05 | D | 1.03 |
| 376 | D | −64.08 | S | 56.4 | S | −3.84 | D | −3.84 |
| 377 | S | −48.87 | D | 51.53 | D | −8.71 | D | −8.71 |
| 378 | S | −33.66 | D | 46.66 | D | −13.58 | S | 6.5 |
| 379 | S | −18.45 | D | 41.79 | D | −18.45 | D | 1.63 |
| 380 | S | −3.24 | S | 57 | S | −3.24 | D | −3.24 |
| 381 | S | 11.97 | D | 52.13 | D | −8.11 | D | −8.11 |
| 382 | S | 27.18 | D | 47.26 | D | −12.98 | S | 7.1 |
| 383 | S | 42.39 | D | 42.39 | D | −17.85 | D | 2.23 |
| 384 | S | 57.6 | S | 57.6 | S | −2.64 | D | −2.64 |
| 385 | D | 52.73 | D | 52.73 | D | −7.51 | D | −7.51 |
| 386 | D | 47.86 | D | 47.86 | D | −12.38 | S | 7.7 |
| 387 | D | 42.99 | D | 42.99 | D | −17.25 | D | 2.83 |
| 388 | D | 38.12 | S | 58.2 | S | −2.04 | D | −2.04 |
| 389 | D | 33.25 | D | 53.33 | D | −6.91 | D | −6.91 |
| 390 | D | 28.38 | D | 48.46 | D | −11.78 | S | 8.3 |
| 391 | D | 23.51 | D | 43.59 | D | −16.65 | D | 3.43 |
| 392 | D | 18.64 | S | 58.8 | S | −1.44 | D | −1.44 |
| 393 | D | 13.77 | D | 53.93 | D | −6.31 | D | −6.31 |
| 394 | D | 8.9 | D | 49.06 | D | −11.18 | S | 8.9 |
| 395 | D | 4.03 | D | 44.19 | D | −16.05 | D | 4.03 |
| 396 | D | −0.84 | S | 59.4 | S | −0.84 | D | −0.84 |
| 397 | D | −5.71 | D | 54.53 | D | −5.71 | D | −5.71 |
| 398 | D | −10.58 | D | 49.66 | D | −10.58 | D | −10.58 |
| 399 | D | −15.45 | D | 44.79 | D | −15.45 | S | 4.63 |
| 400 | D | −20.32 | S | 60 | D | −20.32 | D | −0.24 |
| 401 | D | −25.19 | D | 55.13 | S | −5.11 | D | −5.11 |
| 402 | D | −30.06 | D | 50.26 | D | −9.98 | D | −9.98 |
| 403 | D | −34.93 | D | 45.39 | D | −14.85 | S | 5.23 |
| 404 | D | −39.8 | S | 60.6 | D | −19.72 | D | 0.36 |
| 405 | D | −44.67 | D | 55.73 | S | −4.51 | D | −4.51 |
| 406 | D | −49.54 | D | 50.86 | D | −9.38 | D | −9.38 |
| 407 | D | −54.41 | D | 45.99 | D | −14.25 | S | 5.83 |
| 408 | D | −59.28 | S | 61.2 | D | −19.12 | D | 0.96 |
| 409 | S | −44.07 | D | 56.33 | S | −3.91 | D | −3.91 |
| 410 | S | −28.86 | D | 51.46 | D | −8.78 | D | −8.78 |
| 411 | S | −13.65 | D | 46.59 | D | −13.65 | S | 6.43 |
| 412 | S | 1.56 | S | 61.8 | D | −18.52 | D | 1.56 |
| 413 | S | 16.77 | D | 56.93 | S | −3.31 | D | −3.31 |
| 414 | S | 31.98 | D | 52.06 | D | −8.18 | D | −8.18 |
| 415 | S | 47.19 | D | 47.19 | D | −13.05 | S | 7.03 |
| 416 | S | 62.4 | S | 62.4 | D | −17.92 | D | 2.16 |
| 417 | D | 57.53 | D | 57.53 | S | −2.71 | D | −2.71 |
| 418 | D | 52.66 | D | 52.66 | D | −7.58 | D | −7.58 |
| 419 | D | 47.79 | D | 47.79 | D | −12.45 | S | 7.63 |
| 420 | D | 42.92 | S | 63 | D | −17.32 | D | 2.76 |
| 421 | D | 38.05 | D | 58.13 | S | −2.11 | D | −2.11 |
| 422 | D | 33.18 | D | 53.26 | D | −6.98 | D | −6.98 |
| 423 | D | 28.31 | D | 48.39 | D | −11.85 | S | 8.23 |
| 424 | D | 23.44 | S | 63.6 | D | −16.72 | D | 3.36 |
| 425 | D | 18.57 | D | 58.73 | S | −1.51 | D | −1.51 |
| 426 | D | 13.7 | D | 53.86 | D | −6.38 | D | −6.38 |
| 427 | D | 8.83 | D | 48.99 | D | −11.25 | D | −11.25 |
| 428 | D | 3.96 | S | 64.2 | D | −16.12 | S | 3.96 |
| 429 | D | −0.91 | D | 59.33 | D | −20.99 | D | −0.91 |
| 430 | D | −5.78 | D | 54.46 | S | −5.78 | D | −5.78 |
| 431 | D | −10.65 | D | 49.59 | D | −10.65 | D | −10.65 |
| 432 | D | −15.52 | S | 64.8 | D | −15.52 | S | 4.56 |
| 433 | D | −20.39 | D | 59.93 | D | −20.39 | D | −0.31 |
| 434 | D | −25.26 | D | 55.06 | S | −5.18 | D | −5.18 |
| 435 | D | −30.13 | D | 50.19 | D | −10.05 | D | −10.05 |
| 436 | D | −35 | S | 65.4 | D | −14.92 | S | 5.16 |
| 437 | D | −39.87 | D | 60.53 | D | −19.79 | D | 0.29 |
| 438 | D | −44.74 | D | 55.66 | S | −4.58 | D | −4.58 |
| 439 | D | −49.61 | D | 50.79 | D | −9.45 | D | −9.45 |
| 440 | D | −54.48 | S | 66 | D | −14.32 | S | 5.76 |
| 441 | S | −39.27 | D | 61.13 | D | −19.19 | D | 0.89 |
| 442 | S | −24.06 | D | 56.26 | S | −3.98 | D | −3.98 |
| 443 | S | −8.85 | D | 51.39 | D | −8.85 | D | −8.85 |
| 444 | S | 6.36 | S | 66.6 | D | −13.72 | S | 6.36 |
| 445 | S | 21.57 | D | 61.73 | D | −18.59 | D | 1.49 |
| 446 | S | 36.78 | D | 56.86 | S | −3.38 | D | −3.38 |
| 447 | S | 51.99 | D | 51.99 | D | −8.25 | D | −8.25 |
| 448 | S | 67.2 | S | 67.2 | D | −13.12 | S | 6.96 |
| 449 | D | 62.33 | D | 62.33 | D | −17.99 | D | 2.09 |
| 450 | D | 57.46 | D | 57.46 | S | −2.78 | D | −2.78 |
| 451 | D | 52.59 | D | 52.59 | D | −7.65 | D | −7.65 |
| 452 | D | 47.72 | S | 67.8 | D | −12.52 | S | 7.56 |
| 453 | D | 42.85 | D | 62.93 | D | −17.39 | D | 2.69 |
| 454 | D | 37.98 | D | 58.06 | S | −2.18 | D | −2.18 |
| 455 | D | 33.11 | D | 53.19 | D | −7.05 | D | −7.05 |
| 456 | D | 28.24 | S | 68.4 | D | −11.92 | D | −11.92 |
| 457 | D | 23.37 | D | 63.53 | D | −16.79 | S | 3.29 |
| 458 | D | 18.5 | D | 58.66 | D | −21.66 | D | −1.58 |
| 459 | D | 13.63 | D | 53.79 | S | −6.45 | D | −6.45 |
| 460 | D | 8.76 | S | 69 | D | −11.32 | D | −11.32 |
| 461 | D | 3.89 | D | 64.13 | D | −16.19 | S | 3.89 |
| 462 | D | −0.98 | D | 59.26 | D | −21.06 | D | −0.98 |
| 463 | D | −5.85 | D | 54.39 | S | −5.85 | D | −5.85 |
| 464 | D | −10.72 | S | 69.6 | D | −10.72 | D | −10.72 |
| 465 | D | −15.59 | D | 64.73 | D | −15.59 | S | 4.49 |
| 466 | D | −20.46 | D | 59.86 | D | −20.46 | D | −0.38 |
| 467 | D | −25.33 | D | 54.99 | S | −5.25 | D | −5.25 |
| 468 | D | −30.2 | S | 70.2 | D | −10.12 | D | −10.12 |
| 469 | D | −35.07 | D | 65.33 | D | −14.99 | S | 5.09 |
| 470 | D | −39.94 | D | 60.46 | D | −19.86 | D | 0.22 |
| 471 | D | −44.81 | D | 55.59 | S | −4.65 | D | −4.65 |
| 472 | D | −49.68 | S | 70.8 | D | −9.52 | D | −9.52 |
| 473 | S | −34.47 | D | 65.93 | D | −14.39 | S | 5.69 |
| 474 | S | −19.26 | D | 61.06 | D | −19.26 | D | 0.82 |
| 475 | s | −4.05 | D | 56.19 | S | −4.05 | D | −4.05 |
| 476 | s | 11.16 | S | 71.4 | D | −8.92 | D | −8.92 |
| 477 | s | 26.37 | D | 66.53 | D | −13.79 | S | 6.29 |
| 478 | s | 41.58 | D | 61.66 | D | −18.66 | D | 1.42 |
| 479 | s | 56.79 | D | 56.79 | S | −3.45 | D | −3.45 |
| 480 | s | 72 | S | 72 | D | −8.32 | D | −8.32 |
| 481 | D | 67.13 | D | 67.13 | D | −13.19 | S | 6.89 |
| 482 | D | 62.26 | D | 62.26 | D | −18.06 | D | 2.02 |
| 483 | D | 57.39 | D | 57.39 | S | −2.85 | D | −2.85 |
| 484 | D | 52.52 | S | 72.6 | D | −7.72 | D | −7.72 |
| 485 | D | 47.65 | D | 67.73 | D | −12.59 | S | 7.49 |
| 486 | D | 42.78 | D | 62.86 | D | −17.46 | D | 2.62 |
| 487 | D | 37.91 | D | 57.99 | S | −2.25 | D | −2.25 |
| 488 | D | 33.04 | S | 73.2 | D | −7.12 | D | −7.12 |
| 489 | D | 28.17 | D | 68.33 | D | −11.99 | D | −11.99 |
| 490 | D | 23.3 | D | 63.46 | D | −16.86 | S | 3.22 |
| 491 | D | 18.43 | D | 58.59 | D | −21.73 | D | −1.65 |
| 492 | D | 13.56 | S | 73.8 | S | −6.52 | D | −6.52 |
| 493 | D | 8.69 | D | 68.93 | D | −11.39 | D | −11.39 |
| 494 | D | 3.82 | D | 64.06 | D | −16.26 | S | 3.82 |
| 495 | D | −1.05 | D | 59.19 | D | −21.13 | D | −1.05 |
| 496 | D | −5.92 | S | 74.4 | S | −5.92 | D | −5.92 |
| 497 | D | −10.79 | D | 69.53 | D | −10.79 | D | −10.79 |
| 498 | D | −15.66 | D | 64.66 | D | −15.66 | S | 4.42 |
| 499 | D | −20.53 | D | 59.79 | D | −20.53 | D | −0.45 |
| 500 | D | −25.4 | S | 75 | S | −5.32 | D | −5.32 |

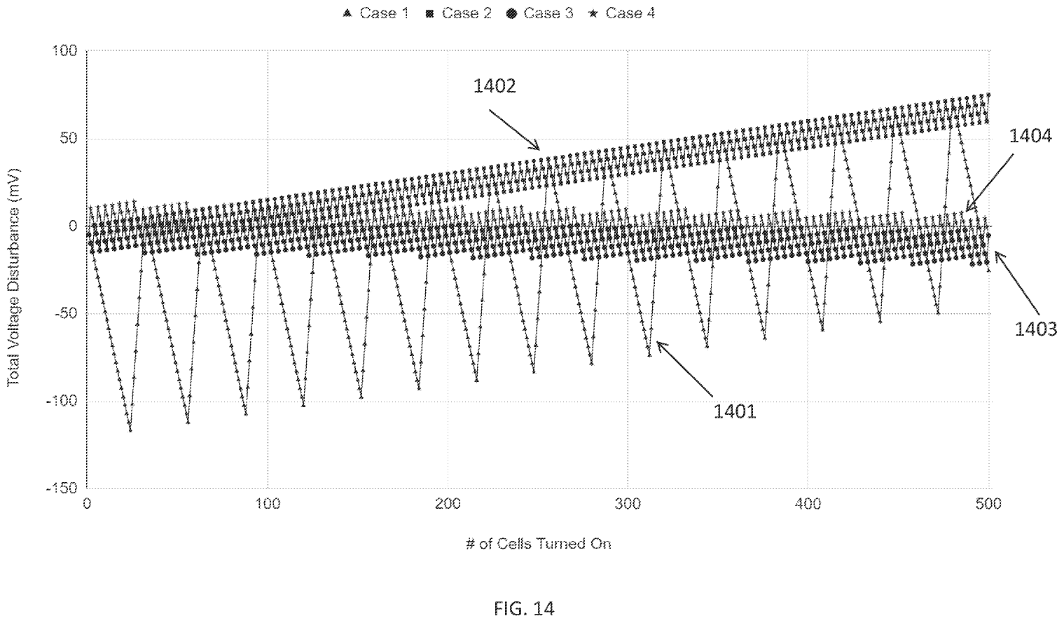

Table 2 illustrates statistics for each of Cases 1, 2, 3, and 4. In particular, Table 2 lists, for each case, the minimum total voltage disturbance value, the maximum total voltage disturbance value, the difference between these two values, and the average total voltage disturbance value (in mV). It should be appreciated that Case 4, in this example, provides an average total voltage disturbance value that is closest 0. In other words, across all the options of how many current source unit cells 114 are turned on, the ordering of Case 4 may provide a total voltage disturbance at V_{bias} that is, on average, closest to 0. FIG. 14 illustrates the information in Table 1 in graph form. Data set 1401 represents the total voltage disturbance as a function of the number of cells that are turned on for case 1. Data set 1402 represents the total voltage disturbance as a function of the number of cells that are turned on for case 2. Data set 1403 represents the total voltage disturbance as a function of the number of cells that are turned on for case 3. Data set 1404 represents the total voltage disturbance as a function of the number of cells that are turned on for case 4. In this plot, the total voltage disturbance is expressed in millivolts (mV). As shown in this figure, cases 3 and 4 are characterized by mean values that are relatively close to 0, especially when compared to cases 1 and 2.

TABLE 2
Statistics for the four cases illustrated in Table 1.

| | Min Value | Max Value | Max-Min | Average |
|---|---|---|---|---|
| Case 1 | −116.88 | 72 | 188.88 | −22.3638 |
| Case 2 | −14.61 | 75 | 89.61 | 30.045 |
| Case 3 | −21.73 | 4.2 | 25.93 | −8.74956 |
| Case 4 | −11.99 | 13.94 | 25.93 | 0.96916 |

In some embodiments, the PLL controller 111 may not include the fine control circuitry 115, and the PLL controller 111 may only control turning on and off of the current source unit cells 114 or 714 in blocks. In some embodiments, the PLL controller 111 may not include the coarse control circuitry 113, and the PLL controller 111 may only control turning on and off of the current source unit cells 114 or 714 with single current source unit cell resolution.

In some embodiments, a method includes using an ultrasound device having any of the PLLs (e.g., the PLL 102 or the PLL 702) described above. As described above, the PLL may include a DCO (e.g., the DCO 110 or the DCO 710) having current source unit cells with drain switch current source unit cells with source switch. Using the ultrasound device may include switching on a certain number of the plurality of current source unit cells with drain switch and a certain number of the plurality of current source unit cells with source switch. Further description of PLLs, DCOs, various example topologies for the current source unit cells, and various methods for switching on the current source unit cells may be found above.

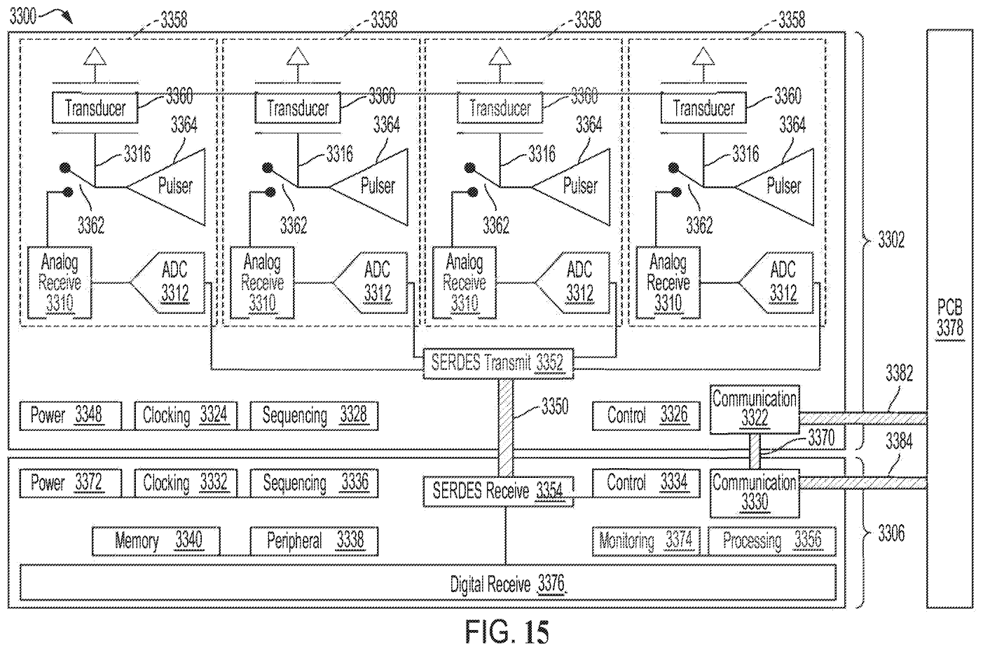

FIG. 15 illustrates a functional block diagram of an exemplary ultrasound-on-chip device 3300, in accordance with certain embodiments described herein. FIG. 15 also illustrates a printed circuit board (PCB) 3378. The ultrasound-on-chip device 3300 includes a first device 3302 and a second device 3306. The first device 3302 and the second device 3306 may each be dies that are packaged together to form the ultrasound-on-chip device 3300. The first device 3302 and the second device 3306 may be application-specific integrated circuits (ASICs). The first device 3302 includes a plurality of elements 3358 (which may also be considered pixels). While only four elements 3358 are shown in FIG. 15, it should be appreciated that many more elements 3358 may be included, such as hundreds, thousands, or tens of thousands of elements. Each of the elements 3358 includes an ultrasonic transducer 3360, a pulser 3364, a receive switch 3362, an analog receive circuitry 3310 block, and an analog-to-digital converter (ADC) 3312. The first device 3302 includes the ultrasonic transducers 3360, the pulsers 3364, the receive switches 3362, the analog receive circuitry 3310, the ADCs 3312, SERDES transmit circuitry 3352, power circuitry 3348, clocking circuitry 3324, sequencing circuitry 3328, control circuitry 3326, and communication circuitry 3322. The second device 3306 includes SERDES receive circuitry 3354, digital receive circuitry 3376, power circuitry 3372, clocking circuitry 3332, sequencing circuitry 3336, control circuitry 3334, communication circuitry 3330, memory circuitry 3340, peripheral management circuitry 3338, monitoring circuitry 3374, and processing circuitry 3356. A communication link 3350 electrically connects the SERDES transmit circuitry 3352 in the first device 3302 to the SERDES receive circuity 3354 in the second device 3306. A communication link 3370 electrically connects the communication circuitry 3322 in the first device 3302 to the communication circuitry 3330 in the second device 3306. A communication link 3382 electrically connects the communication circuitry 3322 in the first device 3302 to the PCB 3378. A communication link 3384 electrically connects the communication circuitry 3330 in the second device 3306 to the PCB 3378.

A pulser 3364 may be configured to output a driving signal to an ultrasonic transducer 3360. The pulser 3364 may receive a waveform from a waveform generator (not shown) and be configured to output a driving signal corresponding to the received waveform. When the pulser 3364 is driving the ultrasonic transducer 3360 (the “transmit phase”), the receive switch 3362 may be open such that the driving signal is not applied to receive circuitry (e.g., the analog receive circuitry 3310).

The ultrasonic transducer 3360 may be configured to emit pulsed ultrasonic signals into a subject, such as a patient, in response to the driving signal received from the pulser 3364. The pulsed ultrasonic signals may be back-scattered from structures in the body, such as blood cells or muscular tissue, to produce echoes that return to the ultrasonic transducer 3360. The ultrasonic transducer 3360 may be configured to convert these echoes into electrical signals. When the ultrasonic transducer 3360 is receiving the echoes (the “receive phase”), the receive switch 3362 may be closed such that the ultrasonic transducer 3360 may transmit the electrical signals representing the received echoes through the receive switch 3362 to the analog receive circuitry 3310. Example ultrasonic transducers 3360 include capacitive micromachined ultrasonic transducers (CMUTs) and piezoelectric micromachined ultrasonic transducers (PMUTs). For example, CMUTs may include cavities formed in a substrate with a membrane/membranes overlying the cavity. The ultrasonic transducers may be arranged in an array (e.g., one-dimensional or two-dimensional).

The analog receive circuitry 3310 may include, for example, one or more analog amplifiers, one or more analog filters, analog beamforming circuitry, analog dechirp circuitry, analog quadrature demodulation (AQDM) circuitry, analog time delay circuitry, analog phase shifter circuitry, analog summing circuitry, analog time gain compensation circuitry, and/or analog averaging circuitry. The analog output of the analog receive circuitry 3310 is outputted to the ADC 3312 for conversion to a digital signal. The digital output of the ADC 3312 is outputted to the SERDES transmit circuitry 3352.

The SERDES transmit circuitry 3352 may be configured to convert parallel digital output of the ADC 3312 to a serial digital stream and to output the serial digital stream at a high-speed (e.g., 2-5 gigabits/second or more) over the communication link 3350. The SERDES receive circuitry 3354 may be configured to convert the serial digital stream received from the communication link 3350 to a parallel digital output and to output this parallel digital output to the digital receive circuitry 3376.

In the ultrasound-on-chip device 3300, one block of SERDES transmit circuitry 3352 receives data from multiple ADC's 3312 and is electrically coupled, through the communication link 3350, to one block of SERDES receive circuitry 3354 that is coupled to the digital receive circuitry 3376. There may be multiple instances of SERDES transmit circuitry 3352, communication link 3350, and SERDES receive circuitry 3354, each receiving data from multiple ADC's 3312. In some embodiments, there may be one instance of SERDES transmit circuitry 3352, communication link 3350, and SERDES receive circuitry 3354 per ADC 3312 and/or per ultrasonic transducer 3360, or more generally, per element 3358. In some embodiments, there may be approximately equal to or between 1-100 parallel instances of SERDES transmit circuitry 3352, communication link 3350, and SERDES receive circuitry 3354. In some embodiments, there may be approximately equal to or between 1-10,000 parallel instances of SERDES transmit circuitry 3352, communication link 3350, and SERDES receive circuitry 3354. The data offload rate of all the parallel instances of SERDES transmit circuitry 3352, communication link 3350, and SERDES receive circuitry 3354 may make the ultrasound-on-chip device 3300 acoustically limited, meaning that it may not be necessary to insert undesired time between collection of frames of ultrasound data to offload data from the ultrasound-on-chip device 3300. The data offload rate may facilitate high pulse repetition intervals (e.g., greater than or equal to approximately 10 kHz).

In some embodiments, the SERDES receive circuitry 3354 may include a mesochronous receiver. In some embodiments, the SERDES receive circuitry 3354 may include a digital PLL, a digital clock and data recovery circuit, and an equalizer. In some embodiments, the PLL of the SERDES receive circuitry 3354 may be implemented using any of the PLL described above. Thus, the PLL may use fast on/off techniques that allow the PLL to power down and conserve power when the ultrasound-on-chip device 3300 is not generating data, and power up to full operating within an acceptably fast period of time when the ultrasound-on-chip device 3300 begins to generate data again. At the same time, the PLL may be configured to prevent large disturbances that may otherwise cause an increase in the settling time of the voltage at a bias node.

The digital receive circuitry 3376 may include, for example, one or more digital filters, digital beamforming circuitry, digital quadrature demodulation (DQDM) circuitry, averaging circuitry, digital dechirp circuitry, digital time delay circuitry, digital phase shifter circuitry, digital summing circuitry, digital multiplying circuitry, requantization circuitry, waveform removal circuitry, image formation circuitry, backend processing circuitry and/or one or more output buffers. The image formation circuitry in the digital receive circuitry 3376 may be configured to perform apodization, back projection and/or fast hierarchy back projection, interpolation range migration (e.g., Stolt interpolation) or other Fourier resampling techniques, dynamic focusing techniques, delay and sum techniques, tomographic reconstruction techniques, Doppler calculation, frequency and spatial compounding, and/or low and high-pass filtering, etc.

Referring to the first device 3302, the communication circuitry 3322 in the first device 3302 may be configured to provide communication between the first device 3302 and the second device 3306 over the communication link 3370 (or more than one communication links 3370). The communication circuitry 3322 may facilitate communication of signals from any circuitry on the first device 3302 to the second device 3306 and/or communication of signals from any circuitry on the second device 3306 to the first device 3302 (aside from communication facilitated by the SERDES transmit circuitry 3352, the communication link 3350, and the SERDES receive circuitry 3354).

The communication circuitry 3322 in the first device 3302 may also be configured to provide communication between the first device 3302 and the PCB 3378 over the communication link 3382 (or more than one communication links 3382). The communication circuitry 3322 may facilitate communication of signals from any circuitry on the first device 3302 to the PCB 3378 and/or communication of signals from any circuitry on the PCB 3378 to the first device 3302. For example, the PCB 3378 may provide control signals to the first device 3302 through the communication link 3382 and the communication circuitry 3322 that may then be used by the control circuitry 3326.

The clocking circuitry 3324 in the first device 3302 may be configured to generate some or all of the clocks used in the first device 3302 and/or the second device 3306. In some embodiments, the clocking circuitry 3324 may receive a high-speed clock (e.g., a 1.5625 GHz or a 2.5 GHz clock) from an external source that the clocking circuitry 3324 may feed to various circuit components of the ultrasound-on-chip device 3300. In some embodiments, the clocking circuitry 3324 may divide and/or multiply the received high-speed clock to produce clocks of different frequencies (e.g., 20 MHz, 40 MHz, 100 MHz, or 200 MHz) that the clocking circuitry 3324 may feed to various components of the ultrasound-on-chip device 3300. In some embodiments, the clocking circuitry 3324 may separately receive two or more clocks of different frequencies, such as the frequencies described above.

The control circuitry 3326 in the first device 3302 may be configured to control various circuit components in the first device 3302. For example, the control circuitry 3326 may control and/or parameterize the pulsers 3364, the receive switches 3362, the analog receive circuitry 3310, the ADCs 3312, the SERDES transmit circuitry 3352, the power circuitry 3348, the communication circuitry 3322, the clocking circuitry 3324, the sequencing circuitry 3328, digital waveform generators, delay meshes, and/or time-gain compensation circuitry (the latter three of which are not shown in FIG. 15). The control circuitry 3326 may also be configured to control any circuitry on the second device 3306.

The sequencing circuitry 3328 in the first device 3302 may be configured to coordinate various circuit components on the first device 3302 that may or may not be digitally parameterized. In some embodiments, the sequencing circuitry 3328 may control the timing and ordering of parameter changes in the first device 3302 and/or the second device 3306, control triggering of transmit and receive events, and control data flow (e.g., from the first device 3302 to the second device 3306). In some embodiments, the sequencing circuitry 3328 may control execution of an imaging sequence which may be specific to the selected imaging mode, preset, and user settings. In some embodiments, the sequencing circuitry 3328 in the first device 3302 may be configured as a master sequencer that triggers events on sequencing circuitry 3336 in the second device 3306 that is configured as a slave sequencer and has been digitally parameterized. In some embodiments, the sequencing circuitry 3336 in the second device 3306 is configured as a master sequencer that triggers events on the sequencing circuitry 3328 in the first device 3302 that is configured as a slave sequencer and has been digitally parameterized. In some embodiments, the sequencing circuitry 3328 in the first device 3302 is configured to control parameterized circuit components on both the first device 3302 and the second device 3306. In some embodiments, the sequencing circuitry 3328 in the first device 3302 and the sequencing circuitry 3336 in the second device 3306 may operate in synchronization by using a clock derived from the same source (e.g., provided by the clocking circuitry).

The power circuitry 3348 in the first device 3302 may include low dropout regulators, switching power supplies, and/or DC-DC converters to supply the first device 3302 and/or the second device 3306. In some embodiments, the power circuitry 3348 may include multi-level pulsers and/or charge recycling circuitry.

The second device 3306 additionally includes communication circuitry 3330, clocking circuitry 3332, control circuitry 3334, sequencing circuitry 3336, peripheral management circuitry 3338, memory circuitry 3340, power circuitry 3372, processing circuitry 3356, and monitoring circuitry 3374. The communication circuitry 3330 in the second device 3306 may be configured to provide communication between the second device 3306 and the first device 3302 over the communication link 3370 (or more than one communication links 3370). The communication circuitry 3330 may facilitate communication of signals from any circuitry on the second device 3306 to the first device 3302 and/or communication of signals from any circuitry on the first device 3302 to the second device 3306.

The communication circuitry 3330 in the second device 3306 may also be configured to provide communication between the second device 3306 and the PCB 3378 over the communication link 3384 (or more than one communication links 3384). The communication circuitry 3330 may facilitate communication of signals from any circuitry on the second device 3306 to the PCB 3378 and/or communication of signals from any circuitry on the PCB 3378 to the second device 3306. For example, the PCB 3378 may provide control signals to the second device 3306 through the communication link 3384 and the communication circuitry 3330 that may then be used by the control circuitry 3334.

The clocking circuitry 3332 in the second device 3306 may be configured to generate some or all of the clocks used in the second device 3306 and/or the first device 3302. In some embodiments, the clocking circuitry 3332 may receive a high-speed clock (e.g., a 1.5625 GHz or a 2.5 GHz clock) that the clocking circuitry 3332 may feed to various circuit components of the ultrasound-on-chip device 3300. In some embodiments, the clocking circuitry 3332 may divide and/or multiply the received high-speed clock to produce clocks of different frequencies (e.g., 20 MHz, 40 MHz, 100 MHz, or 200 MHz) that the clocking circuitry 3332 may feed to various components. In some embodiments, the clocking circuitry 3332 may separately receive two or more clocks of different frequencies, such as the frequencies described above.

The control circuitry 3334 in the second device 3306 may be configured to control various circuit components in the second device 3306. For example, the control circuitry 3334 may control and/or parameterize the SERDES receive circuitry 3354, the digital receive circuitry 3376, the communication circuitry 3330, the clocking circuitry 3332, the sequencing circuitry 3336, the peripheral management circuitry 3338, the memory circuitry 3340, the power circuitry 3372, and the processing circuitry 3356. The control circuitry 3334 may also be configured to control any circuitry on the first device 3302.

The sequencing circuitry 3336 in the second device 3306 may be configured to coordinate various circuit components on the second device 3306 that may or may not be digitally parameterized. In some embodiments, the sequencing circuitry 3336 in the second device 3306 is configured as a master sequencer that triggers events on the sequencing circuitry 3328 in the first device 3302 that has been digitally parameterized. In some embodiments, the sequencing circuitry 3328 in the first device 3302 is configured as a master sequencer that triggers events on the sequencing circuitry 3336 in the first device 3302 that is configured as a slave sequencer and has been digitally parameterized. In some embodiments, the sequencing circuitry 3336 in the second device 3306 is configured to control parameterized circuit components on both the first device 3302 and the second device 3306. In some embodiments, the sequencing circuitry 3336 in the second device 3306 and the sequencing circuitry 3328 in the first device 3302 may operate in synchronization by using a clock derived from the same source (e.g., provided by the clocking circuitry).

The peripheral management circuitry 3338 may be configured to generate a high-speed serial output data stream. For example, the peripheral management circuitry 3338 may be a Universal Serial Bus (USB) 2.0, 3.0, or 3.1 module. The peripheral management circuitry 3338 may additionally or alternatively be configured to allow an external microprocessor to control various circuit components of the ultrasound-on-chip device 3300 over a USB connection. As another example, the peripheral management circuitry 3338 may include a WiFi module or a module for controlling another type of peripheral. In some embodiments, this high-speed serial output data stream may be outputted to the PCB 3378.

The memory circuitry 3340 may be configured to buffer and/or store digitized image data (e.g., image data produced by imaging formation circuitry and/or other circuitry in the digital receive circuitry 3376). For example, the memory circuitry 3340 may be configured to enable the ultrasound-on-chip device 3300 to retrieve image data in the absence of a wireless connection to a remote server storing the image data. Furthermore, when a wireless connection to a remote server is available, the memory circuitry 3340 may also be configured to provide support for wireless connectivity conditions such as lossy channels, intermittent connectivity, and lower data rates, for example. In addition to storing digitized image data, the memory circuitry 3340 may also be configured to store timing and control parameters for synchronizing and coordinating operation of elements in the ultrasound-on-chip device 3300. The power circuitry 3372 may include power supply amplifiers for supplying power to the second device 3306.

The processing circuitry 3356, which may be in the form of one or more embedded processors, may be configured to perform processing functions. In some embodiments, the processing circuitry 3356 may be configured to perform sequencing functions, either for the first device 3302 or for the second device 3306. For example, the processing circuitry 3356 may control the timing and ordering of parameter changes in the first device 3302 and/or the second device 3306, control triggering of transmit and receive events, and/or control data flow (e.g., from the first device 3302 to the second device 3306). In some embodiments, the processing circuitry 3356 may control execution of an imaging sequence which may be specific to the selected imaging mode, preset, and user settings. In some embodiments, the processing circuitry 3356 may perform external system control, such as controlling the peripheral management circuitry 3338, the processing circuitry 3356, controlling power sequencing (e.g., for the power circuitry 3348 and/or the power circuitry 3372), and interfacing with the monitoring circuitry 3374. In some embodiments, the processing circuitry 3356 may perform internal system control, such as configuring data flow within the chip (e.g., from the first device 3302 to the second device 3306), calculating or controlling the calculation of processing and image formation parameters (e.g., for image formation circuitry), controlling on chip clocking (e.g., for the clocking circuitry 3324 and/or the clocking circuitry 3332), and/or controlling power (e.g., for the power circuitry 3348 and/or the power circuitry 3372). The processing circuitry 3356 may be configured to perform functions described above as being performed by other components of the ultrasound-on-chip device 3300, and in some embodiments certain components described herein may be absent if their functions are performed by the processing circuitry 3356.

The monitoring circuitry 3374 may include, but is not limited to, temperature monitoring circuitry (e.g., thermistors), power measurement circuitry (e.g., voltage and current sensors), nine-axis motion circuitry (e.g., gyroscopes, accelerometers, compasses), battery monitoring circuitry (e.g., coulomb counters), and/or circuitry checking for status or exception conditions of other on-board circuits (e.g., power controllers, protection circuitry, etc.).

It should be understood that there may be many more instances of each component shown in FIG. 15. For example, there may be hundreds, thousands, or tens of thousands of ultrasonic transducers 3360, pulsers 3364, receive switches 3362, analog receive circuitry 3310 blocks, SERDES transmit circuitry 3352 blocks, SERDES receive circuitry 3354 blocks, and/or digital receive circuitry 3376 blocks. Additionally, it should be understood that certain components shown in FIG. 15 may receive signals from more components than shown or transmit signals to more components than shown (e.g., in a multiplexed fashion, or after averaging). For example, a given pulser 3364 may output signals to one or more ultrasonic transducers 3360, a given receive switch 3362 may receive signals from one or more ultrasonic transducers 3360, a given block of analog receive circuitry 3310 may receive signals from one or more receive switches 3362, a given ADC 3312 may receive signals from one or more blocks of analog receive circuitry 3310, a given block of SERDES transmit circuitry 3352 may receive signals from one or more ADCs 3312. In some embodiments, a given ultrasound element may have an ultrasonic transducer 3360 and a dedicated pulser 3364, receive switch 3362, analog receive circuitry 3310 block, ADC 3312, and/or SERDES transmit circuitry 3352 block. It should also be understood that certain embodiments of an ultrasound-on-chip device may have more or fewer components than shown in FIG. 15.

According to an aspect of the present application, an ultrasound device is provided, comprising a phase-locked loop (PLL) comprising a digitally-controlled oscillator (DCO) comprising a plurality of current source unit cells with respective drain switches and a plurality of current source unit cells with respective source switches.

In some embodiments, the plurality of current source unit cells with respective drain switches have a first circuit topology, the plurality of current source unit cells with respective source switches have a second circuit topology, and the first and second circuit topologies are different.

In some embodiments, the PLL is configured to use a fast switching technique that allows the PLL to power down when the ultrasound device is not generating data and to power up within 1 microsecond from when the ultrasound device begins to generate data again

In some embodiments, switching on one of the plurality of current source unit cells with respective drain switches causes a voltage transition at an internal node of the current source unit cell with respective drain switches proceeding in a first voltage direction, switching on of the plurality of current source unit cells with respective source switches causes a voltage transition at an internal node of the current source unit cell with respective source switches proceeding in a second voltage direction, and the first and second voltage directions are opposite.

In some embodiments, each of the plurality of current source unit cells with respective drain switches comprises a switch, and a current source wherein the current source comprises a first terminal and a second terminal and the switch is coupled to the first terminal of the current source and each of the plurality of current source unit cells with respective source switches comprises the switch and the current source wherein the switch is coupled to the second terminal of the current source.

In some embodiments, the current source comprises a single transistor, the first terminal comprises a drain of the transistor, and the second terminal comprises a source of the transistor.

In some embodiments, the current source comprises a cascode current source that comprises multiple transistors, the first terminal comprises a drain of one of the multiple transistors, and the second terminal comprises a source of the one of the multiple transistors.

In some embodiments, the switch comprises a single transistor.

In some embodiments, the switch comprises a transmission gate.

In some embodiments, the current source comprises at least one transistor having a gate terminal, and the gate terminal is coupled to an output of a resistor-capacitor (RC) filter.

In some embodiments, the RC filter is coupled to an output terminal of bias generation circuitry.

In some embodiments, the switch comprises at least one transistor having a gate terminal, the PLL comprises a decoder having a plurality of output terminals; and the gate terminal of the transistor is coupled to one of the plurality of output terminals of the decoder.

In some embodiments, the DCO comprises a ring oscillator and each of the plurality of current source unit cells with respective drain switches and each of the plurality of current source unit cells with respective source switches is couplable to the ring oscillator.

In some embodiments, the ring oscillator is configured to generate a clock signal having a frequency that depends on an amount of current that the ring oscillator receives from the plurality of current source unit cells with respective drain switches and the plurality of current source unit cells with respective source switches.

In some embodiments, the ultrasound device is configured to control the frequency of the clock signal, at least in part, by switching on a certain number of the plurality of current source unit cells with respective drain switches and a certain number of the plurality of current source unit cells with respective source switches.

In some embodiments, the ultrasound device may further comprise control circuitry comprising coarse control circuitry configured to control how many blocks of one or more of the plurality of current source unit cells with respective drain switches and/or one or more of the plurality of current source unit cells with respective source switches are turned on.

In some embodiments, each of the plurality of current source unit cells with respective drain switches and each of the plurality of current source unit cells with respective source switches is coupled to an output terminal of a resistor-capacitor (RC) filter, a ratio between a decrease in voltage at the output terminal of the RC filter caused by one of the plurality of current source unit cell with respective drain switches switching on to an increase in voltage at the output terminal of the RC filter caused by one of the current source unit cell with respective source switches switching on is m:n, each or approximately each of the blocks comprises 1+floor (n/m) current source unit cells with a composition of floor (n/m) current source unit cells with respective drain switches and one current source unit cell with respective source switches.

According to an aspect of the present application, a method is provided, the method comprising using an ultrasound device comprising a phase-locked loop (PLL) comprising a digitally-controlled oscillator (DCO) comprising a plurality of current source unit cells with respective drain switches. and a plurality of current source unit cells with respective source switches, wherein using the ultrasound device comprises switching on a certain number of the plurality of current source unit cells with respective drain switches and a certain number of the plurality of current source unit cells with respective source switches.

In some embodiments, the plurality of current source unit cells with respective drain switches have a first circuit topology, the plurality of current source unit cells with respective source switches have a second circuit topology, and the first and second circuit topologies are different.

In some embodiments, the PLL is configured to use a fast switching technique that allows the PLL to power down when the ultrasound device is not generating data and to power up within 1 microsecond when the ultrasound device begins to generate data again.

In some embodiments, switching on one of the plurality of current source unit cells with respective drain switches causes a voltage transition at an internal node of the current source unit cell with respective drain switches proceeding in a first voltage direction, switching on of the plurality of current source unit cells with respective source switches causes a voltage transition at an internal node of the current source unit cell with respective source switches proceeding in a second voltage direction, and the first and second voltage directions are different.

In some embodiments, each of the plurality of current source unit cells with respective drain switches comprises a switch, and a current source wherein the current source comprises a first terminal and a second terminal and the switch is coupled to the first terminal of the current source and each of the plurality of current source unit cells with respective source switches comprises the switch, and the current source wherein the switch is coupled to the second terminal of the current source.

In some embodiments, the current source comprises a single transistor, the first terminal comprises a drain of the transistor, and the second terminal comprises a source of the transistor.

In some embodiments, the current source comprises a cascode current source that comprises multiple transistors, the first terminal comprises a drain of one of the multiple transistors, and the second terminal comprises a source of the one of the multiple transistors.

In some embodiments, the switch comprises a single transistor.

In some embodiments, the switch comprises a transmission gate.

In some embodiments, the current source comprises at least one transistor having a gate terminal, and the gate terminal is coupled to an output of a resistor-capacitor (RC) filter.

In some embodiments, the RC filter is coupled to an output terminal of bias generation circuitry.

In some embodiments, the switch comprises at least one transistor having a gate terminal, the PLL comprises a decoder having a plurality of output terminals and the gate terminal of the transistor is coupled to one of the plurality of output terminals of the decoder.

In some embodiments, the DCO comprises a ring oscillator, and each of the plurality of current source unit cells with respective drain switches and each of the plurality of current source unit cells with respective source switches is couplable to the ring oscillator.

In some embodiments, using the ultrasound device comprises using the ring oscillator is configured to generate a clock signal having a frequency that depends on an amount of current that the ring oscillator receives from the plurality of current source unit cells with respective drain switches and the plurality of current source unit cells with respective source switches.

In some embodiments, using the ultrasound device comprises controlling the frequency of the clock signal, at least in part, by switching on the certain number of the plurality of current source unit cells with respective drain switches and the certain number of the plurality of current source unit cells with respective source switches.

In some embodiments, the ultrasound device further comprises control circuitry comprising coarse control circuitry and using the ultrasound device comprises using the coarse control circuitry to switch on or off of blocks comprising one or more of the plurality of current source unit cells with respective drain switches and or more of the plurality of current source unit cells with respective source switches at a time.

In some embodiments, each of the plurality of current source unit cells with respective drain switches and each of the plurality of current source unit cells with respective source switches is coupled to an output terminal of a resistor-capacitor (RC) filter, the RC filter is coupled to an output terminal of bias generation circuitry, a ratio between a decrease in voltage at the output terminal of the RC filter caused by one of the plurality of current source unit cell with respective drain switches switching on to an increase in voltage at the output terminal of the RC filter caused by one of the current source unit cell with respective source switches on is m:n, each or approximately each of the blocks comprises 1+floor (n/m) current source unit cells with a composition of floor (n/m) current source unit cells with respective drain switches and one current source unit cell with respective source switches.

According to an aspect of the present application, an ultrasound device is provided, the device comprising a plurality of ultrasound transducers, serializer-deserializer (SerDes) circuitry coupled to the plurality of ultrasound transducers, and a phase-locked loop (PLL) comprising a plurality of current source unit cells of a first type and a plurality of current source unit cells of a second type different from the first type.

In some embodiments, the at least one of the plurality of current source unit cells of the first type is configured to produce a first voltage increase when turned on, and wherein at least one of the plurality of current source unit cells of the second type is configured to produce a first voltage decrease when turned on.

In some embodiments, the at least one of the plurality of current source unit cells of the first type is configured to produce a second voltage decrease when turned off, and wherein the at least one of the plurality of current source unit cells of the second type is configured to produce a second voltage increase when turned off.

In some embodiments, the least one of the plurality of current source unit cells of the first type comprises a drain switch and at least one of the plurality of current source unit cells of the second type comprises a source switch.

In some embodiments, the source switch comprises a transmission gate.

In some embodiments, the source switch comprises a cascode current source.

According to an aspect of the present application, a method is provided for operating an ultrasound device, the method comprising producing a plurality of electric signals using a plurality of ultrasound transducers, combining the plurality of electric signals using serializer-deserializer (SerDes) circuitry, wherein the combining comprises timing the SerDes circuitry using a phase-locked loop (PLL) at least in part by turning on a plurality of current source unit cells of a first type and at least in part by turning on a plurality of current source unit cells of a second type different from the first type.

In some embodiments, turning on the plurality of current source unit cells of the first type and turning on the plurality of current source unit cells of the second type comprises determining how many current source unit cells of the first type and how many current source unit cells of the second type should be turned on to limit a voltage disturbance.

In some embodiments, determining how many current source unit cells of the first type and how many current source unit cells of the second type should be turned on to limit the voltage disturbance comprises identifying a plurality of entries of a look-up table, that, collectively, yield a voltage disturbance below a predefined threshold.

While the above description has described various circuitry and methods for operating such circuitry in the context of ultrasound devices, the circuitry and methods may be used in the context of other electronic devices as well.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

As used herein, reference to a numerical value being between two endpoints should be understood to encompass the situation in which the numerical value can assume either of the endpoints. For example, stating that a characteristic has a value between A and B, or between approximately A and B, should be understood to mean that the indicated range is inclusive of the endpoints A and B unless otherwise noted.

The terms “approximately” and “about” may be used to mean within ±20% of a target value in some embodiments, within ±10% of a target value in some embodiments, within ±5% of a target value in some embodiments, and yet within ±2% of a target value in some embodiments. The terms “approximately” and “about” may include the target value.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Having described above several aspects of at least one embodiment, it is to be appreciated various alterations, modifications, and improvements will readily occur to those skilled in the art. Such alterations, modifications, and improvements are intended to be object of this disclosure. Accordingly, the foregoing description and drawings are by way of example only.

What is claimed is:

1. An ultrasound device comprising:
a phase-locked loop (PLL) comprising:
a digitally-controlled oscillator (DCO) comprising:
a plurality of current source unit cells with respective drain switches, each of the plurality of current source unit cells with respective drain switches comprising:
a first switch; and
a first current source comprising one or more first transistors;
wherein:
the first switch is coupled to a drain terminal of one of the one or more first transistors of the first current source; and
a plurality of current source unit cells with respective source switches, each of the plurality of current source unit cells with respective source switches comprising:
a second switch; and
a second current source comprising one or more second transistors;
wherein:
the second switch is coupled to a source terminal of one of the one or more second transistors of the second current source.

2. The ultrasound device of claim 1, wherein the PLL is configured to use a fast switching technique that allows the PLL to power down when the ultrasound device is not generating data and/or allows the PLL to power up within 1 microsecond from when the ultrasound device begins to generate data again.

3. The ultrasound device of claim 1, wherein switching on one of the plurality of current source unit cells with respective drain switches causes a voltage transition at an internal node of the current source unit cell with respective drain switches proceeding in a first voltage direction, switching on of the plurality of current source unit cells with respective source switches causes a voltage transition at an internal node of the current source unit cell with respective source switches proceeding in a second voltage direction, and the first voltage direction is opposite the second voltage direction.

4. The ultrasound device of claim 1, wherein:
the first current source comprises a single first transistor, and the first switch is coupled to a drain terminal of the single first transistor; and/or
the second current source comprises a single second transistor, and the second switch is coupled to a source terminal of the single second transistor.

5. The ultrasound device of claim 1, wherein:
the first current source comprises a first cascode current source that comprises multiple first transistors, and the first switch is coupled to a drain terminal of one of the multiple first transistors; and/or
the second current source comprises a second cascode current source that comprises multiple second transistors, and the second switch is coupled to a source terminal of one of the multiple second transistors.

6. The ultrasound device of claim 1, wherein:
the first switch comprises a first single transistor; and/or
the second switch comprises a second single transistor.

7. The ultrasound device of claim 1, wherein:
the first switch comprises a first transmission gate; and/or
the second switch comprises a second transmission gate.

8. The ultrasound device of claim 1, wherein:
the first current source comprises at least one transistor having a first gate terminal;
the second current source comprises at least one transistor having a second gate terminal;
the first gate terminal of each of the plurality of current source unit cells with respective drain switches is coupled to an output of a resistor-capacitor (RC) filter; and
the second gate terminal of each of the plurality of current source unit cells with respective source switches is coupled to the output of the RC filter.

9. The ultrasound device of claim 8, wherein the RC filter is coupled to an output terminal of bias generation circuitry.

10. The ultrasound device of claim 1, wherein:
the first switch comprises at least one transistor having a first gate terminal;
the second switch comprises at least one transistor having a second gate terminal;
the PLL comprises a decoder having a plurality of output terminals;
the first gate terminal of each of the plurality of current source unit cells with respective drain switches is coupled to one of the plurality of output terminals of the decoder; and
the second gate terminal of each of the plurality of current source unit cells with respective source switches is coupled to one of the plurality of output terminals of the decoder.

11. The ultrasound device of claim 1, wherein:
the DCO comprises a ring oscillator; and
each of the plurality of current source unit cells with respective drain switches and each of the plurality of current source unit cells with respective source switches is couplable to the ring oscillator.

12. The ultrasound device of claim 11, wherein the ring oscillator is configured to generate a clock signal having a frequency that depends on an amount of current that the ring oscillator receives from the plurality of current source unit cells with respective drain switches and the plurality of current source unit cells with respective source switches.

13. The ultrasound device of claim 12, wherein the ultrasound device is configured to control the frequency of the clock signal, at least in part, by switching on a certain number of the plurality of current source unit cells with respective drain switches and a certain number of the plurality of current source unit cells with respective source switches.

14. The ultrasound device of claim 1, further comprising control circuitry comprising coarse control circuitry configured to control how many blocks of one or more of the plurality of current source unit cells with respective drain switches and/or one or more of the plurality of current source unit cells with respective source switches are turned on.

15. The ultrasound device of claim 14, wherein:
each of the plurality of current source unit cells with respective drain switches and each of the plurality of current source unit cells with respective source switches is coupled to an output terminal of a resistor-capacitor (RC) filter;
a ratio between a decrease in voltage at the output terminal of the RC filter caused by one of the plurality of current source unit cell with respective drain switches switching on to an increase in voltage at the output terminal of the RC filter caused by one of the current source unit cell with respective source switches switching on is m:n;
each or approximately each of the blocks comprises 1+floor (n/m) current source unit cells with a composition of floor (n/m) current source unit cells with respective drain switches and one current source unit cell with respective source switches.

16. An ultrasound device comprising:
a plurality of ultrasound transducers;
serializer-deserializer (SerDes) circuitry coupled to the plurality of ultrasound transducers; and
a phase-locked loop (PLL) comprising a plurality of current source unit cells of a first type and a plurality of current source unit cells of a second type different from the first type.

17. The ultrasound device of claim 16, wherein at least one of the plurality of current source unit cells of the first type is configured to produce a first voltage increase when turned on, and wherein at least one of the plurality of current source unit cells of the second type is configured to produce a first voltage decrease when turned on.

18. The ultrasound device of claim 17, wherein the at least one of the plurality of current source unit cells of the first type is configured to produce a second voltage decrease when turned off, and wherein the at least one of the plurality of current source unit cells of the second type is configured to produce a second voltage increase when turned off.

19. The ultrasound device of claim 16, wherein at least one of the plurality of current source unit cells of the first type comprises a drain switch and at least one of the plurality of current source unit cells of the second type comprises a source switch.

20. A method for operating an ultrasound device, the method comprising:
producing a plurality of electric signals using a plurality of ultrasound transducers;
combining the plurality of electric signals using serializer-deserializer (SerDes) circuitry, wherein the combining comprises:
timing the SerDes circuitry using a phase-locked loop (PLL) at least in part by turning on a plurality of current source unit cells of a first type and at least in part by turning on a plurality of current source unit cells of a second type different from the first type.

21. The method of claim 20, wherein turning on the plurality of current source unit cells of the first type and turning on the plurality of current source unit cells of the second type comprises determining how many current source unit cells of the first type and how many current source unit cells of the second type should be turned on to limit a voltage disturbance.

22. The method of claim 21, wherein determining how many current source unit cells of the first type and how many current source unit cells of the second type should be turned on to limit the voltage disturbance comprises identifying a plurality of entries of a look-up table, that, collectively, yield a voltage disturbance below a predefined threshold.
